SEQUENCE LISTING (12 sequences)

SEQ ID NO: 1 - protein, 15 aa, Artificial Sequence
  peptide; amino acid residues 12-26 of human p53 protein
PPLSQETFSDLWKLL

SEQ ID NO: 2 - protein, 9 aa, Artificial sequence
  peptide; amino acid residues 12-20 of human p53 protein
PPLSQETFS

SEQ ID NO: 3 - protein, 10 aa, Artificial sequence
  peptide; amino acid residues 17-26 of human p53 protein
ETFSDLWKLL

SEQ ID NO: 4 - protein, 17 aa, Artificial sequence
  peptide; penetratin leader sequence from antennapedia
KKWKMRRNQFWVKVQRG

SEQ ID NO: 5 - DNA, 62 bp, Artificial sequence
  primer
atccggtaccaaatggagaccttttctgacctctggaaactcctctagaagcggccgcactc

SEQ ID NO: 6 - DNA, 62 bp, Artificial sequence
  primer
taggccatggtttacctctggaaaagactggagacctttgaggagatcttcgccggcgtgag

SEQ ID NO: 7 - DNA, 38 bp, Artificial sequence
  primer
atccggcccagccggccgcgctcctgctgtgcttcgtg

SEQ ID NO: 8 - DNA, 33 bp, Artificial sequence
  primer
atccgcggccgcagcgcgatttgaaggagggac

SEQ ID NO: 9 - DNA, 12 bp, Artificial sequence
  primer
atccgcggccgc

SEQ ID NO: 10 - DNA, 10 bp, Artificial sequence
  primer
atcccctagg

SEQ ID NO: 11 - DNA, 32 bp, Artificial sequence
  primer
atccggatcctggtatggagacagacacactc

SEQ ID NO: 12 - DNA, 29 bp, Artificial sequence
  primer
atccctcgagctttccagcttggtccccc

(19) United States
(12) Patent Application Publication
(10) Pub. No.: US 2016/0083475 A1
(43) Pub. Date: Mar. 24, 2016

(54) PANCREATIC CANCER ASSOCIATED ANTIGEN, ANTIBODY THERETO, AND DIAGNOSTIC AND TREATMENT METHODS

(71) Applicants: Josef Michl, Little Neck, NY (US); Stefan M. Bradu, Rego Park, NY (US); Raquib Hannan, East Meadow, NY (US); Matthew R. Pincus, Brooklyn, NY (US)

(72) Inventors: Josef Michl, Little Neck, NY (US); Stefan M. Bradu, Rego Park, NY (US); Raquib Hannan, East Meadow, NY (US); Matthew R. Pincus, Brooklyn, NY (US)

(21) Appl. No.: 14/492,408

(22) Filed: Sep. 22, 2014

(51) Int. Cl. C07K 16/30; A61K 47/48; G01N 33/574

(52) U.S. Cl. CPC C07K 16/303; G01N 33/57438; A61K 47/486; A61K 47/48438; G01N 2333/705; C07K 2317/734; A61K 2039/505

(57) ABSTRACT

The present invention is directed to an antigen found on the surface of rat and human pancreatic cancer cells and provides antibodies of high specificity and selectivity to this antigen as well as hybridomas secreting the subject antibodies. Methods for both the diagnosis and treatment of pancreatic cancer are also provided. This tissue marker of pancreatic adenocarcinoma, an approximately 43.5 kD surface membrane protein designated PaCa-Ag1, is completely unexpressed in normal pancreas but abundantly expressed in pancreatic carcinoma cells. Moreover, a soluble form of PaCa-Ag1 exists, having a molecular weight about 36 to about 38 kD, that is readily identified in sera and other body fluids of pancreatic cancer patients, using a subject antibody.

### CROSS REFERENCE TO RELATED APPLICATIONS

This application is a divisional application of U.S. application Ser. No. 10/542,239, filed Mar. 3, 2006; which is a 371 application of PCT/2004/001196, having an international filing date of Jan. 16, 2004; which claims benefit to U.S. Provisional Application Ser. No. 60/440,699, filed Jan. 17, 2003.

### BACKGROUND OF THE INVENTION

1. Field of the Invention

The present invention resides in the discovery of a specific antigen found on the surface of pancreatic carcinoma cells and monoclonal antibodies of high specificity and selectivity to the antigen. Both the antigen and antibodies thereto may be used in diagnosing and treating pancreatic cancer in an animal, especially a human.

2. Description of the Related Art

Pancreatic cancer is a nearly always fatal disease with a median survival time of only 80-90 days for a patient diagnosed with the disease. Pancreatic cancer is one of the more lethal forms of cancer in numbers of patients killed in the U.S. Less than 4% of patients are alive 5 years from the time of diagnosis, and none after approximately 7 years. At present, no pancreatic cancer-specific markers, pancreatic cancer-specific antibodies, nor pancreatic cancer-specific assays exist that identify a pancreatic cancer-specific antigen in bodily fluids or secretions.

One reason that pancreatic cancer (PaCa) claims 29,000 new lives every year in the U.S. alone and, therefore, occupies the fourth position in the cancer-related mortality hierarchy, is the lack of an early diagnostic tool. An effective early diagnostic tool requires a marker that is specific for PaCa and can be identified at a time when therapeutic intervention is successful in preventing progression of the lethal disease.

A cost-effective, non-invasive test for detecting pancreatic carcinoma at early, curable stages is urgently needed. Only 8% of patients have local disease, compared to 51% with distant disease at the time of diagnosis (Jemal 2003); the former have a 5 year survival of 17-30%, compared to 2% for the latter (Jemal 2003, Yeo 1995). The extremely high mortality rate, non-resectability of 85% of pancreatic lesions at the time of clinical symptomatic presentation, the lack of any effective therapy and the fact that even lesions 2 cm or less (usually discovered incidentally) may have already metastasized or may still have a high mortality rate, pose daunting challenges for development of a useful test for early detection of pancreatic malignancy (Birkmeyer et al 1999, Russell 1990, Nix et al 1991, Tsuchiya et al 1986). The cost to society for pancreatic adenocarcinoma has been estimated to be $2.6 billion per year for treatment alone (Elixhauser and Halpern, 1999); this figure does not take into account lost earnings and other factors impacted by the morbidity and mortality of this disease.

Presently, the only widely used clinical serologic test for diagnosing pancreatic carcinoma and monitoring disease progression and response to therapy is the ELISA assay for Carbohydrate Antigen 19-9 (CA 19-9). The CA19-9 detected by a monoclonal antibody made against a colon carcinoma cell line antigen (Koprowski et al, 1979) is a ganglioside sialyl-lacto-N-fucopentaose (Magnani et al, 1982) that is expressed at high levels in many pancreatic adenocarcinomas, but is also present in cells in the normal pancreas, biliary and gastrointestinal tract (Arends 1982, Rollhauser and Steinberg 1998). Hence, inflammation or damage to these tissues results in spillage of CA19-9 into the bloodstream, leading to false positive elevations in common non-neoplastic disorders such as pancreatitis, cirrhosis and obstructive cholangitis (Rollhauser and Steinberg 1998). The false positivity of the CA19-9 ELISA has been reported to range from 2 to 54% (Jalanko et al 1984, Eskelinen and Haglund 1999), rendering the CA19-9 assay useless as a screen for early detection of pancreatic adenocarcinoma. Furthermore, CA19-9 is also elevated in a spectrum of non-pancreatic malignancies including cholangiocarcinoma, hepatocellular carcinoma, carcinomas of the gastrointestinal tract (colon, stomach, esophagus) and several other cancers (Steinberg 1990, Maestranzi et al 1998, Carpelan-Holmstrom et al 2002).

The sensitivity of CA19-9 has been reported to range from 68 to 93% using the recommended cut-off value of 37 U/ml (Steinberg 1990, Jalanko et al 1984, Eskelinin and Haglund 1999). The sensitivity drops significantly for detection of resectable versus unresectable lesions; in one representative study, the sensitivity for the latter was 90%, dropping to 74% for detection of resectable lesions (Safi et al. 1998). The CA19-9 oligosaccharidc chain also defines the Lewis' blood group antigen (Magnani et al. 1992). Approximately 10-15% of the population do not express this antigen (Tempero et al. 1987), rendering CA19-9 useless in this subpopulation not only for early detection but also for monitoring response to therapy and relapse via reduction and elevation in CA19-9 (exceptions being a small number of Lewis^{a}-negative patients with pancreatic cancer expression of the CA19-9 antigen (Yazawa et al, 1987; Takasaki et al, 1988, von Rosen et al, 1993).

Another more recently discovered molecular target on pancreatic carcinoma cells with clinical diagnostic potential as a serologic marker is the phosphatidylinositol-linked surface protein mesothelin (Chang et al., 1992), which is overexpressed in the vast majority of pancreatic adenocarcinomas (Argani et al 2001). Mesothelin is expressed on normal mesothelial cells and is present in 95% of ovarian adenocarcinomas (tumors derived from modified mesothelial cells on the ovarian surface) in mesotheliomas, and a significant number of non-small cell lung carcinomas, breast, endometrial, cervical, endometrial, gastric and colon carcinomas (Chang and Pastan, 1994; Scholler et al, 1999).

One technology that has been proposed for early detection of pancreatic carcinoma involves detection of aberrant DNA from stool samples. The method has been promoted for early detection of adenocarcinoma of the colon and demonstrated in pancreatic adenocarcinoma in a few small studies (Caldas, 1994). A serologic diagnostic assay that detects an antigen specific to pancreatic cancer cells but is completely unexpressed in normal pancreas, and which is not found (or is found only in trace amounts) in other tissue, could prove to be far more effective than the CA19-9 immunoassay or mesothelin marker.

The present invention is directed to the discovery of a pancreatic carcinoma-specific antigen, designated 3C4-Ag (or PaCa-Ag1). This antigen, is primarily localized on the surface of rat and human pancreatic cancer cells and as tested to date, is not detected in normal, untransformed cells except for trace amounts in normal ovary. Thus, the present invention represents a much needed improvement in the area of pancreatic cancer detection and treatment. The PaCa-Ag1 antigen is also present in sera and other bodily fluids of pancreatic carcinoma patients. In addition, the present invention is also directed to antibodies which specifically bind to the PaCa-Ag1 antigen. The subject antigen and antibodies are useful in both methods of diagnosis and treatment of pancreatic cancer, also provided herein.

### SUMMARY OF THE INVENTION

In accordance with the present invention, there is provided a pancreatic carcinoma-specific antigen 3C4-Ag (PaCa-Ag1) in substantially purified form. 3C4-Ag may be characterized by a molecular weight of about 43 or 43.5 kDa as determined by SDS-PAGE; a pI on isoelectrofocusing of about 4.5 to about 5.0; and the absence of significant glycosylation. 3C4-Ag is primarily localized on the surface of rat and human pancreatic cancer cells and is not detected in normal, non-proliferating cells. The PaCa-Ag1 antigen is also present in sera and other bodily fluids of pancreatic cancer patients but is not present in the blood or sera of healthy individuals. Immunologically active fragments of 3C4-Ag are also encompassed by the present invention.

Antibodies or binding portions thereof, having binding specificity to pancreatic carcinoma specific antigen 3C4-Ag are also provided wherein said antigen is characterized by a molecular weight of about 43 or 43.5 kDa as determined by SDS-PAGE; a pI on isoelectrofocusing of about 4.5 to about 5.0; the absence of significant glycosylation; and being primarily localized on the surface of rat and human pancreatic cancer cells and in the sera of pancreatic cancer patients but not detected in normal, non-proliferating cells or sera from healthy individuals. Subject antibodies may be polyclonal or monoclonal and may also be in a humanized form. In addition, a subject antibody may be labeled with a fluorophore, chemilophore, chemiluminecer, photosensitizer, suspended particles, radioisotope or enzyme. In another embodiment, a subject antibody may be conjugated or linked to a diagnostic, therapeutic drug, or toxin.

The present invention also provides Murine hybridoma cell lines which produce monoclonal antibodies specifically immunoreactive with the 3C4-Ag antigen.

In another aspect of the invention, there is provided a method of detecting pancreatic cancer in an animal subject. The method comprises the steps of: (a) contacting a cell, tissue or fluid sample from the subject with at least one of an antibody or binding portion thereof which specifically binds to 3C4-Ag or an immunologically active fragment thereof, the monoclonal antibody mAb3C4; or an antibody which binds the epitope bound by the monoclonal antibody mAb3C4, or an antibody which binds another epitope on the 3C4 antigen protein; under conditions permitting said antibody to specifically bind an antigen in the sample to form an antibody-antigen complex; (b) detecting antibody-antigen complexes in the sample; and (c) correlating the detection of elevated levels of antibody-antigen complexes in the sample compared to a control sample with the presence of pancreatic cancer.

In still another embodiment of the invention, there is provided a diagnostic kit suitable for detecting 3C4-Ag in a cell, tissue, or fluid sample from a patient. The kit may comprise a number of different components such as: (a) an antibody or binding portion thereof which specifically binds 3C4-Ag or an immunologically active fragment thereof, (b) a conjugate of a specific binding partner for the antibody or binding portion thereof; and (c) a label for detecting the bound antibody.

In another aspect of the invention, a method of treating pancreatic cancer in a patient is provided. The method comprises the steps of administering to the patient an effective amount of an antibody or binding portion thereof which specifically binds to 3C4-Ag or an immunologically active fragment thereof, wherein said antibody or binding portion thereof is conjugated or linked to a therapeutic drug or toxin.

A pharmaceutical composition comprising an antibody or binding portion thereof which specifically binds to 3C4-Ag, admixed with a pharmaceutically acceptable carrier is also provided. The antibody or binding portion thereof which specifically binds to 3C4-Ag may be conjugated or linked to a therapeutic drug or toxin in the pharmaceutical composition.

### BRIEF DESCRIPTION OF THE FIGURES

FIGS. 1A-1F are photomicrographs showing morphological changes induced by NNK in BMRPA1 cells. FIG. 1A shows normal appearance of untreated BMRPA1 cells. FIGS. 1B through 1F show sequential cell passages (1-12) after one 16 h treatment of BMRPA1 with NNK.

FIGS. 2A-2C are photomicrographs of immunofluorescence (IF) stained live BMRPA1.NNK cells with ISHIP mice serum (A), with 3C4 hybridoma spent medium (B) and normal, untransformed BMRPA1 cells with 3C4 hybridoma spent medium (C). The surface expression of the 3C4-Ag on BMRPA1.NNK cells is clearly apparent in FIG. 2B in the linear ring-like fluorescence image while the BMRPA1 cells are completely devoid of any staining.

FIG. 3 is a photograph of a stained SDS-PA gel run with G-protein affinity purified mAb3C4 from ascites. Lane 1: hybridoma injected mouse ascites; Lane 2: low pH eluation where IgG was quantitatively released from the heads. Lane 3 shows the ˜160 kD protein (IgG) of lane 2 reduced. Lanes 1B and 2B depict immunoblots and autoradiograms (chemiluminescentograms) of the IgG in lanes 1 and 2 using HRP-SaM IgG and ECL reaction kit, confirming the ˜160 kD protein to be IgG.

FIG. 4 is an autoradiograph showing SDS PAGE of cell lysate proteins from rodent and human pancreatic carcinoma cells, followed by an immunoblot with mAb3C4.

FIG. 5A is gel photograph showing silver stained lysates of BMRPA1.NNK cells processed without mAb3C4 (lane 1) and with mAb3C4 and protein G beads (lane 2).

FIG. 5B is an immunoblot for the 3C4-Ag in the immunoprecipitates from the lysates in FIG. 5A (BMRPA1.NNK cells). Immunoprecipitate obtained (lane 1) without mAb3C4, IB with mAb3C4 and HRP-SαM IgG; (lane 2) with mAb3C4, IB with mAb3C4 and HRP-SαM IgG identifying the 3C4-Ag as 43 kD polypeptide; (lane 3) with mAb3C4, IB without mAb3C4 but with HRP-SαM IgG.

FIGS. 6A, 6C, 6E, 6G, and 6I are phase contrast visible light photomicrographs of live rodent and human pancreas carcinoma cells stained with mAb3C4, FIGS. 6B, 6D, 6F, 6H, and 6J are UV light photographs processed identically and showing membrane fluorescence. FIGS. 6A and 6B: BMRPA1.NNK cells; FIGS. 6C and 6D: BMRPA1.TUC3 cells; FIGS. 6E and 6F: CAPAN-1 cells; FIGS. 6G and 6H: CAPA2-2 cells; FIGS. 6I and 6J are BxPC3 cells. FIGS. 6A-6D are rodent pancreatic carcinoma cells. FIGS. 6E-6J are human pancreatic carcinoma cells.

FIGS. 7A-7C show Fluorescent Activated Cell Sorting (FAC'S) analysis of transformed and untransformed rodent and human PaCa cells. FIG. 7A: BMRIPA1.Tuc3; FIG. 7B: BMRPA1.NNK; FIG. 7C: human MIA PaCa; all showing fluorescence intensity of the selected cell population. Peaks labeled (1) indicate background fluorescence by processing the cells with FITC-RαMIgG only (no primary antibody)(background control): (2) cells reacted with mAb3C4 and FITC-RαMIgG.

FIG. 8 graphically depicts cytotoxicity of mAb3C4 in the presence of active complement. X axis: rabbit serum (complement) dilutions; Y axis: percentage of cells alive after exposure to mAb3C4 and rabbit complement. The first bar of each group shows treatment of cells with fresh rabbit serum only (source of active complement) for 45 minutes at 37° C. The second bar of each group represents cells treated with mAb3C4 and fresh rabbit strum (source of active complement) for 45 minutes at 37° C. The third bar of the first group represents cells treated with mAb3C4 followed by heat inactivated (30-45 minutes at 56° C.) rabbit serum (inactivated complement).

FIGS. 9A-9B are immunoblots of tissue extracts using mAb3C4; FIG. 9A: rat; FIG. 9B: human. Reduced proteins from extracts from various tissues (thyroid, ovary, brain, heart, lung, liver, testes. FIG. 9A) as well as human acinar pancreatic cells, white blood cells, and ductal pancreatic cells were separated on 12% SDS PAGE, electrophoretically transferred to nitrocellulose and processed with and without mAb3C4 followed by ECL chemiluminescence amplification. MIA-PaCa and mouse IgG served as controls. “+” means reaction with primary mAb. “−” means no reaction with primary mAb. MIA-PaCa and mouse IgG served as positive controls. “*” indicates tissue extract was obtained by Dounze homogenization in the presence of Triton X-100 containing lysing buffer. “#” indicates tissue extract was obtained by high frequency pulse sonication in the presence of Triton X-100 containing lysing buffer.

FIG. 10 shows autoradiographs of immunoblots of various cancerous human tissues using mAb3C4.

FIG. 11 is a gel photo of proteins of BMRPA1.NNK cell lysates separated by two dimensional gel (2-D-Gel) electrophoresis according to size and pI, and identified by silver staining.

FIG. 12 is a chemiluminescentogram showing the proteins of BMRPA1.NNK cell lysates separated by 2D-Gel-electrophoresis as described for FIG. 11, electrophoretically transferred to PVDF membrane and blotted with mAb3C4. The arrow indicates the location of the 3C4 antigen.

FIG. 13 graphically depicts the effect of in vivo administration of mAb3C4 on tumor growth.

FIGS. 14A-14B are FACS analysis of mAb34C binding to PaCa-Ag1 on BMRPA1.TUC3 cells without (FIG. 15A) and with (FIG. 15B) trypsin treatment. Open peak in FIG. 14A=non-specific IgG staining (background).

FIG. 15 is a photograph of an SDS page gel and is the control to FIG. 16. FIG. 15 shows that parallel deglycosylation of fetuin (˜51 kD) results in smaller polypeptides of 43-45 kD, indicating the intact enzymatic activity during the incubation conditions used in parallel for the deglycosylation of the PaCa-Ag1 protein in FIG. 16.

FIG. 16 is an immunoblot demonstrating enzymatic deglycosylation of PaCa-Ag1 does not change the molecular weight of the polypeptide.

FIGS. 17A-17B graphically depict Antibody-Antigen adsorbance ELISA for PaCa-Ag1. The titration of mAb3C4 concentration of semi-pure PAcA-Ag1 is shown in FIG. 17A. In FIG. 17B, PaCaAg1 is present in spent cell culture media.

FIGS. 18A-18B graphically depict Antibody-Antigen adsorbance ELISA for PaCa-Ag1. FIG. 18A shows the presence of PaCaAg1 in ascites of mice xenotransplanted with pancreatic carcinoma BMRPA.TUC3 cells. FIG. 18B shows PaCa-Ag1 in pancreatic duct juice of pancreatic cancer patient.

FIG. 19 is an immunoblot blot with mAB3C4 of serum proteins from patients confirmed with pancreatic cancer and from a healthy volunteer. Lanes 1, 2, and 3 were loaded with individual serum samples from 3 pancreatic cancer patients. Arrows in these lanes point to the reaction product of mAb3C4 with a polypeptide of about 36-38 kD. Lane 4 was loaded with a serum sample from healthy volunteer. Lane 5 was loaded with a healthy volunteer sample spiked with an equal amount of PaCa-Ag1 positive serum of patient of lane 3. Arrow in Lane 4 points to absence of reaction product. Arrow in Lane 5 points to reaction product.

### DETAILED DESCRIPTION OF THE INVENTION

The present invention is directed to a pancreatic carcinoma-specific antigen and antibodies which specifically bind thereto. The pancreatic carcinoma-specific antigen (pancreatic cancer associated antigen), also referred to hereinafter interchangeably as 3C4-Ag or PaCa-Ag1, has a molecular weight of about 43 or 43.5 kDa as determined by SDS polyacrylamide electrophoresis (SDS PAGE) and is primarily localized on the surface of pancreatic cancer cells. 3C4-Ag is not detected in normal, non-proliferating cells and is only detected at very low levels in renal, prostate and possibly colon carcinoma.

The present invention is also directed to a soluble form of 3C4-Ag (PaCa-Ag1) present in, and isolatable from, sera or other bodily fluids of pancreatic cancer patients and having a molecular weight of about 35 kDa.

3C4-Ag was initially identified by indirect immuno-fluorescence (IF) on intact, live and intact, fixed pancreatic cancer cells (rat and human cell lines) as a cell surface antigen, using a mouse monoclonal antibody, mAbC4, as a primary antibody, followed by fluorescein-labeled sheep or rabbit anti-mouse IgG (FITC-S or R anti-M IgG) and fluorescence microscopy. The monoclonal antibody mAb3C4 was produced using an immunosubstractive-hyperimmunization protocol (ISHIP), which protocol is fully described in Applicants' Provisional Patent Application, entitled “Tolerance-Induced Targeted Antibody Production (TITAP).” U.S. Ser. No. 60/413,703, filed Jan. 29, 2003, the disclosure of which is incorporated by reference herein as if fully set forth. In accordance with the ISHIP protocol, cyclophosphamide-induced tolerance in a mouse to antigens present on untransformed rat pancreatic cells (BMRP1 cells) followed by subsequent hyper-immunizations with BMRPA1 cells neoplastically transformed with the known carcinogen 4-(methyl-nitrosamino)-1-(3-pyridyl)-1-butanone (hereinafter BMRP1.NNK cells), resulted in increased immigration of plasma cells secreting antibodies to BMRPA1.NNK cells into the spleen of the mouse. Subsequent fusion of splenocytes from immunized mice with P3U1 myeloma cells resulted in the production of hybridomas secreting antibodies which specifically react with a pancreatic cancer associated antigen (3C4-Ag) on the surface of BMRPA1.NNK, but not untransformed cells.

In accordance with the present invention, there is provided a pancreatic carcinoma specific antigen 3C4-Ag in substantially purified form. The 3C4-Ag is characterized by: a molecular weight of about 43 or 43.5 kDa as determined by SDS-PAGE; a pI on isoelectrofocusing of about 4.5 to about 5.0; and by the absence of significant glycosylation; and being soluble in 50 mM Tris-HCl, 1% NP40, 0.5% sodium deoxycholate, 0.1% SDS, 5 mM EDTA, 1 μg/mL, pepstatin, 2 ug/mL aprotinin, 1 mM PMSF, and 5 mM iodoacetamide; and being primarily localized on the surface of rat and human pancreatic cancer cells but not detected in normal, untransformed cells.

Also in accordance with the present invention, there is provided an antibody having binding specificity to pancreatic carcinoma specific antigen 3C4-Ag, wherein said antigen is characterized by a molecular weight of about 43 or 43.5 kDa as determined by SDS-PAGE; a pI on isoelectrofocusing of about 4.5 to about 5.0; and being soluble in 50 mM Tris-HCl, 1% NP40, 0.5% sodium deoxycholate, 0.1% SDS, 5 mM EDTA, 1 μg/mL pepstatin, 2 ug/mL aprotinin, 1 mM PMSF, and 5 mM iodoacetamide; and being primarily localized on the surface of rat and human pancreatic cancer cells but not detected in normal, untransformed cells. A subject antibody which specifically binds to 3C4-Ag may be a polyclonal or monoclonal antibody. Preferably, the antibody is a monoclonal antibody (mAb). Even more preferably, the mAb is 3C4.

The antibody described above also has binding specificity to a pancreatic carcinoma specific antigen 3C4-Ag, wherein said antigen is in soluble form and isolatable from the sera or other bodily fluids of pancreatic cancer patients.

A murine hybridoma cell line which produces a monoclonal antibody specifically immunoreactive with 3C4-Ag is also provided. Preferably, the murine hybridoma cell line produces mAb3C4.

The pancreatic cancer associated antigen 3C4-Ag, may be prepared using a number of well known methods. 3C4-Ag may be identified and its gene sequence obtained using an immunosubtractive hybridization or differential RNA display methodology. A gene encoding the 3C4-Ag under control of a promoter which functions in a particular host cell may be used to transfect such a host cell in order to express the antigen. Alternatively, 3C4-Ag may be chemically synthesized using well known methods.

Pancreatic cancer associated antigen 3C4-Ag may be purified using well known methods in the art such as polyacrylamide gel electrophoresis (PAGE: see, e.g., Harrington, M. G. (1990) Methods Enzymol., 182:488-495), and size-exclusion chromatography. Other purification techniques, such as immunoaffinity chromatography using an antibody which binds 3C4-Ag such as mAb3C4, may also be performed. Such methods are exemplified herein in Example 8. Following SDS PAGE, the 3C4-Ag band of about 43 kDa may be excised from the gel and eluted into an appropriate buffer. Further purification of 3C4-Ag may be performed including gel filtration, ion exchange chromatography and/or high performance liquid chromatography (HPLC). HPLC is the preferred method of purification.

Purified 3C4-Ag or an immunologically active fragment thereof, may be used to inoculate an animal in order to produce polyclonal antibodies which react with 3C4-Ag. By “immunologically active fragment” is meant a fragment of the approximately 43 or 43.5 kDa 3C4-Ag protein which fragment is sufficient to stimulate production of antibodies which specifically react with an exposed epitope on 3C4-Ag as 3C4-Ag is exposed on the surface of pancreatic cancer cells or which react with the soluble form of 3C4-Ag isolatable from the sera or other bodily fluids of pancreatic cancer patients. Thus, in addition to mAb3C4, the present invention contemplates other antibodies, polyclonal or monoclonal, which specifically react with 3C4-Ag or an immunologically active fragment thereof and which antibodies may or may not bind to the same epitope on 3C4-Ag as does mAb3C4.

Animals, for example, mammals such as mice, goats, rats, sheep or rabbits, or other animals such as poultry, e.g., chickens, can be inoculated with 3C4-Ag or immunologically active fragment thereof, preferably conjugated with a suitable carrier protein to produce polyclonal antibodies. Such immunizations may, be repeated as necessary at intervals of up to several weeks in order to obtain a sufficient titer of antibodies. Blood is collected from the animal to determine if antibodies are produced, the antiserum is tested for response to the 3C4-Ag or immunologically active fragment thereof, and reboosting is undertaken, as needed. In some instances, after the last antigen boost, the animal is sacrificed and spleen cells removed. Immunoglobulins are purified from the serum obtained from the immunized animals. These immunoglobulins can then be utilized in diagnostic immunoassays to detect the presence of antigen in a sample, or in therapeutic applications.

Preferably, monoclonal antibodies which specifically react against 3C4-Ag or immunologically active fragment thereof are prepared. Methods of producing monoclonal antibodies are well known in the art such as described in Kohler and Milstein (1975) Nature 256:495-497, which is incorporated by reference herein as if fully set forth. For example, an animal may be immunized with 3C4-Ag or immunologically active fragment thereof, and spleen cells from the immunized animal obtained. The antibody-secreting lymphocytes are then fused with mycloma cells or transformed cells which are capable of replicating indefinitely in cell culture. Resulting hybridomas may be cultured and the resulting colonies screened for the production of the desired monoclonal antibodies. Antibody producing colonies may be grown either in vivo or in vitro in order to produce large amounts of antibody.

The hybridoma cell line may be propagated in vitro, and the culture medium containing high concentrations of the mAb (such as mAb3C4) harvested by decantation, filtration, or centrifugation. Alternatively, a sample of a subject antibody such as mAb3C4 may be injected into a histocompatible animal of the type used to provide the somatic and myeloma cells for the original fusion. e.g., a mouse. Tumors secreting the mAb develop in the injected animal and body fluids of the animal, such as ascites, fluid, or serum produce mAb in high concentrations.

Fusion with mammalian mycloma cells or other fusion partners capable of replicating indefinitely in cell culture is effected by standard and well-known techniques, for example, by using polyethylene glycol (PEG) or other fusing agents such as described in Milstein and Kohler (1976) Eur. J. Immunol. 6:511, Brown et al. (1981) J. Immunol. 127(2):539-46, Brown et al. (1980) J. Biol. Chem., 255:4980-83, and Yeh et al., Proc. Nat'l. Acad. Sci. (USA) 76(6):2927-31, which disclosures are incorporated by reference herein as if fully set forth. Such an immortal cell line is preferably murine, but may also be derived from cells of other mammalian species such as rats and human. Preferably, the cell line is deficient in enzymes necessary for the utilization of certain nutrients, is capable of rapid growth and has a good fusion capability. Such cell lines are known to those skilled in the art.

Methods for purifying monoclonal antibodies include ammonium sulfate precipitation, ion exchange chromatography, and affinity chromatography such as described in Zola et al. in Monoclonal Hybridoma Antibodies: Techniques and Applications, Hurell (ed) pp. 5-52 (CRC Press 1982) the disclosure of which is incorporated by reference herein as if fully set forth. As described in the present application, Example 7, mice may be injected with 3C4 hybridoma cells, followed by collection of ascites. mAb3C4 may be purified from the ascites using G-protein affinity beads. After washing the beads in an appropriate buffer, the bound mAb3C4 may be eluted from the beads with an elution buffer and separated by the beads by brief centrifugation.

In addition to utilizing whole antibodies, the methods of the present invention encompass use of binding portions of antibodies which specifically bind 3C4-Ag or an immunologically active fragment thereof. Such binding portions include Fab fragments. F(ab′)2 fragments, and Fe fragments. These antibody fragments may be made by conventional procedures, such as proteolytic fragmentation procedures, as described in Goding. Monoclonal Antibodies: Principles and Practice, pp. 98-118, New York. Academic Press (1983), which is incorporated by reference herein as if fully set forth.

The present invention also provides diagnostic methods for detecting pancreatic cancer in a patient. The diagnostic methods are based on immunoassays which detect the presence of pancreatic carcinoma specific antigen (3C4-Ag) in a sample from a patient by reacting with a subject antibody which specifically binds 3C4-Ag or an immunologically active fragment thereof. Examples of patient sample sources include cells, tissue, tissue lysate, tissue extract, or blood-derived sample (such as blood, serum, or plasma), urine, or feces. Preferably, the sample is fluid. The fluid sample is preferably blood serum but could be other fluids such as pleural or aseitie fluid. A detected increase in the level of 3C4-Ag in a sample correlates with a diagnosis of pancreatic cancer in the patient.

There are many different types of immunoassays which may be used in the methods of the present invention. Any of the well known immunoassays may be adapted to detect the level of 3C4-Ag in a serum sample or other sample of a patient, which reacts with an antibody which specifically binds 3C4-Ag, such as, e.g., enzyme linked immunoabsorbent assay (ELISA), fluorescent immunosorbent assay (FIA), chemical linked immunosorbent assay (CLIA), radioimmuno assay (RIA), and immunoblotting (IB). For a review of the different immunoassays which may be used, see: The Immunoassay Handbook, David Wild, ed., Stockton Press, New York, 1994; Sikora et al. (eds.), Monoclonal Antibodies, pp. 32-52, Blackwell Scientific Publications (1984).

For example, an immunoassay to detect pancreatic cancer in a patient involves contacting a sample from a patient with a first antibody or binding portion thereof (e.g., mAb3C4), which is preferably soluble and detectable to form an antibody-antigen complex with 3C4-Ag in the sample. The complex is contacted with a second antibody which recognizes constant regions of the heavy chains of the first antibody. For example, the second antibody may be an antibody which recognizes constant regions of the heavy chains of mouse immunoglobulin which has reacted with mAb3C4 (anti-mouse antibody). The second antibody is labeled with a fluorophore, chemilophore, chemiluminescer, photosensitizer, suspended particles, or radioisotope. Free labeled second antibody is separated from bound antibody. The signal generated by the sample is then measured depending on the signal producing system used. Increased optical density or radioactivity when compared to samples from normal patients correlates with a diagnosis of pancreatic cancer in a patient.

Alternatively, an enzyme-labeled antibody such as e.g., 3-galactosidase-labeled antibody, is used and an appropriate substrate with which the enzyme label reacts is added and allowed to incubate. Enzymes may be covalently linked to 3C4-Ag reactive antibodies for use in the methods of the invention using well known conjugation methods. For example, alkaline phosphatase and horseradish peroxidase may be conjugated to antibodies using glutaraldehyde. Horseradish peroxidase may also be conjugated using the periodate method. Commercial kits for enzyme conjugating antibodies are widely available. Enzyme conjugated anti-human and anti-mouse immunoglobulin specific antibodies are available from multiple commercial sources.

Enzyme labeled antibodies produce different signal sources, depending on the substrate. Signal generation involves the addition of substrate to the reaction mixture. Common peroxidase substrates include ABTS® (2,2′-azinobis(ethylbenzothiazoline-6-sulfonate)), OPD (O-phenylenediamine) and TMB (3,3′,5,5′-tetramethylbenzidine). These substrates require the presence of hydrogen peroxide, p-nitrophenyl phosphate is a commonly used alkaline phosphatase substrate. During an incubation period, the enzyme gradually converts a proportion of the substrate to its end product. At the end of the incubation period, a stopping reagent is added which stops enzyme activity. Signal strength is determined by measuring optical density, usually via spectrophotometer.

Alkaline phosphatase labeled antibodies may also be measured by fluorometry. Thus in the immunoassays of the present invention, the substrate 4-methylumbelliferyl phosphate (4-UMP) may be used. Alkaline phosphatase dephosphorylates 4-UMP to form 4-methylumbelliferone (4-MU), the fluorophore. Incident light is at 365 nm and emitted light is at 448 nm.

As an alternative to enzyme-labeled antibodies, fluorescent compounds, such as fluorescein, rhodamine, phycoerytherin, indocyanine, biotin, phycocyanine, cyanine 5, cyanine 5.5, cyanine 7, cyanine 3, aminomethyl cumarin (AMCA), peridinin chlorophyl, Spectral red, or Texas red may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic color visually detectable with a light microscope. As in the EIA, the fluorescent labeled antibody is allowed to bind to the first antibody-hapten complex. After washing off the unbound reagent, the remaining ternary complex is then exposed to the light of the appropriate wavelength. The fluorescence observed indicates the presence of the hapten of interest, in this case 3C4-Ag. Immunofluorescence and EIA techniques are both very well established in the art and are particularly preferred for the present method. However, other reporter molecules, such as radioisotope, chemiluminescent or bioluminescent molecules, may also be employed. It will be readily apparent to the skilled technician how to vary the procedure to suit the required purposes.

A subject antibody may also be detected with a group of secondary labeled ligands which are capable of binding to the antibody. For example, using conventional techniques biotin may be linked to antibodies produced according to the present invention. The biotinylated antibody is then allowed to contact and bind 3C4-Ag. Streptavidin or avidin which has been labeled with a known label is then contacted with the antibody 3C4-Ag complex which then leads to binding of the labeled streptavidin or avidin to the biotin portion of the biotinylated antibody. Additional biotin may be added followed by the addition of more labeled streptavidin or avidin. Since each streptavidin or avidin molecule is capable of binding tour biotin molecules, a relatively large three-dimensional network is created which includes numerous labels which may be detected by conventional fluorescence microscopy or by radiographic techniques.

Other immunoassay techniques are available for utilization in the present invention as shown by reference to U.S. Pat. Nos. 4,016,043; 4,424,279; and 4,018,653. This, of course, includes both single-site and two-site, or “sandwich”, assays of the non-competitive types, as well as the traditional competitive binding assays described above. A number of variations of the sandwich assay technique exist, and all are intended to be encompassed by the present invention.

In the typical forward sandwich assay, a first antibody having specificity for 3C4-Ag or an immunologically active fragment thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, heads, discs or microplates, or any other surface suitable for conducting an immunoassay. The binding processes are well-known in the art and generally consist of cross-linking, covalently binding, or physically adsorbing the molecule to the insoluble carrier. Following binding, the polymer-antibody complex is washed in preparation for the test sample. An aliquot of the sample to be tested is then added to the solid phase complex and incubated for a period of time sufficient to allow binding to the antibody. The incubation period will vary, but will generally be in the range of about 2-40 minutes. Following the incubation period, the antibody subunit solid phase is washed and dried and incubated with a second antibody specific for a portion of the hapten. The second antibody is linked to a reporter molecule which is used to indicate the binding of the second antibody to the hapten.

Variations on the forward assay include a simultaneous assay, in which both sample and labeled antibody are added simultaneously to the bound antibody, or a reverse assay in which the labeled antibody and sample to be tested are first combined, incubated and then added to the unlabeled surface bound antibody. These techniques are well known to those skilled in the art, and the possibility of minor variations will be readily apparent to those skilled in the art.

Cross-linkers suitable for use in coupling a label to an antibody are well-known. Homofunctional and heterobifunctional cross-linkers are all suitable. Reactive groups which can be cross-linked with a cross-linker include primary amines, sulfhydryls, carbonyls, carbohydrates and carboxylic acids. Cross-linkers are available with varying lengths of spacer arms or bridges. Cross-linkers suitable for reacting with primary amines include homobifunctional cross-linkers such as imidoesters and N-hydroxysuccinimidyl (NHS) esters.

Heterobifunctional cross-linkers which possess two or more different reactive groups are suitable for use herein. Examples include cross-linkers which are amine-reactive at one end and sulfhydryl-reactive at the other end such as 4-succinimidyl-oxycarbonyl-α-(2-pyridyldithio)-toluene, N-succinimidyl-3-(2-pyridyldithio)-propionate and maleimide cross-linkers.

The amount of color, fluorescence, luminescence, or radioactivity present in the reaction (depending on the signal producing system used) is proportionate to the amount of 3C4-Ag in a patient's sample which reacts with a subject antibody such as mAb3C4. Quantification of optical density may be performed using spectrophotometric methods. Quantification of radiolabel signal may be performed using scintillation counting. Increased levels of 3C4-Ag reacting with a subject antibody such mAb3C4 over normal sample levels correlate with a diagnosis of pancreatic cancer in the patient.

The present invention also provides diagnostic kits for performing the methods described hereinabove. In one embodiment, the diagnostic kit comprises: (i) an antibody or binding portion thereof, which specifically binds to 3C4-Ag or an immunologically active fragment thereof, (ii) a conjugate of a specific binding partner for the antibody, and (iii) a label for detecting the bound antibody. In a preferred embodiment, the antibody which specifically binds to 3C4-Ag is mAb3C4. An example of a conjugate of a specific binding partner for mAb3C4 is an antibody which specifically binds to mAb3C4. If the label is an enzyme, then a third container, containing a substrate for the enzyme may be provided.

The kit may also comprise other components such as buffering agents and protein stabilizing agents, e.g., polysaccharides, and the like. In addition, a subject kit may comprise other agents of the signal-producing system such as agents for reducing background interference, control reagents, and compositions suitable for conducting the diagnostic test. Such compositions may include for example, solid surfaces such as glass or polymer such as cellulose, polyacrylamide, nylon, polystyerene, polyvinyl chloride or polypropylene. Solid supports may be in the form of tubes, beads, discs, or microplates, or any other surface for conducting an immunoassay.

The antibodies of the present invention are also useful for in vivo diagnostic applications for the detection of pancreatic tumors, preferably human. For example, pancreatic tumors may be detected by tumor imaging techniques using mAb34C labeled with an appropriate imaging reagent that produces detectable signal. Imaging reagents and procedures for labeling antibodies with such reagents are well known. See e.g., Wensel and Meares, Radio Immunoimaging and Radioimmunotherapy, Esevier, New York (1983); Colcher et al., Meth. Enzymol. 121:802-816 (1986). The labeled antibody may then be detected by e.g., radionuclear scanning as described in Bradwell et al. Monoclonal Antibodies for Cancer Detection and Therapy, Baldwin et al. (eds), pp. 65-85, Academic Press (1985).

In accordance with the present invention, there are also provided therapeutic methods for treating a patient suffering from pancreatic cancer. For example, the mAb3C4 may be used alone to target tumor cells or used in conjunction with an appropriate therapeutic agent to treat pancreatic cancer. When a subject antibody which binds 3C4-Ag or an immunologically active fragment thereof, is used alone, such treatment can be effected by initiating endogenous host immune functions, such as complement-mediated or antibody-dependent cellular cylotoxicity (ADCC). ADCC involves an antibody which can kill cancer cells in the presence of human lymphocytes or macrohages or becomes cytotoxic to tumor cells in the presence of human complement. An antibody of the present invention, which specifically reacts with 3C4-Ag may be modified for ADCC using techniques developed for the production of chimeric antibodies as described by Oi et al., (1986) Biotechnologies 4(3):214-221; and Fell et al. (1989) Proc. Natl. Acad. Sci. USA 86:8507-8511.

In a preferred embodiment, a subject antibody which specifically binds 3C4-Ag or an immunologically active fragment thereof, may be conjugated or linked to a therapeutic drug or toxin for delivery of the therapeutic agent to the site of cancer. Enzymatically active toxins and fragments thereof include but are not limited to: diptheria toxin A fragment, nonbonding active fragments of diptheria toxin, exotoxin A from Pseudomonas aeruginosa, ricin A chain, abrin A chain, modeccin A chain, α-sacrin, certain Aleurites fordii proteins, certain Dianthin proteins. Phytolacca americana proteins (PAP, PAPII and PAP-S), Morodica charantia inhibitor, curcin, crotin, Saponaria offcinalis inhibitor, gelonin, mitogillin, restrictocin, phenomycin, enomycin, and derivatives (including synthetic) of taxol, for example. International Patent Publications WO 84/03508 and WO 85/03508, incorporated by reference herein as if fully set forth, describe procedures for preparing enzymatically active polypeptides of such immunotoxins.

Other cytotoxic moieties include but are not limited to those derived from adriamycin, chlorambucil, daunomycin, methotrexate, neocarzinostatin, and platinum. Procedures for conjugating chlorambucil with antibodies are described in Flechner (1973) European J. Cancer 9:741-745; Ghose et al. (1972) British Medical J. 3:495-499, and Szekerke et al., (1972) Neoplasma 19:211-215, which are incorporated by reference herein as if fully set forth. Procedures for conjugating daunomycin and adriamycin to antibodies are described in Hurwitz et al. (1975) Cancer Research 35:1175-1181 and Arnon et al., (1982) Cancer Surveys 1:429-449, the disclosures of which are also incorporated by reference herein as if fully set forth. Procedures for preparing antibody-ricin conjugates are described e.g., in U.S. Pat. No. 4,414,148 and in Osawa et al., (1982) Cancer Surveys 1:373-388 as well as the references cited therein, which are incorporated by reference herein as if fully set forth. European Patent Application 86309516.2 also describes coupling procedures and is incorporated by reference herein.

A group of peptides has recently been discovered to be especially cytotoxic to pancreatic cancer cells. See copending U.S. patent application Ser. No. 10/386,737, filed Mar. 12, 2003, and applications cited therein (U.S. Provisional Application Ser. No. 60/363,785, filed Mar. 12, 2002; U.S. Ser. No. 09/827,683, filed Apr. 5, 2001, and U.S. Ser. No. 60/195,102, filed Apr. 5, 2000), the disclosures of which are incorporated by reference herein as if fully set forth. These toxic peptides comprise a sequence of amino acids within the p53 protein. p53 protein is a protein of 393 amino acids and is a vital regulator of the cell cycle. Absence of the p53 protein is associated with cell transformation and malignant disease. Haffner, R&Oren, M. (1995) Curr. Opin. Genet. Dev. 5:84-90.

As described in U.S. Ser. No. 10/386,737 and parent applications cited therein, peptides toxic to pancreatic cancer cells may be derived from a peptide having the following amino acid sequence: PPLSQETFSDLWKLL (SEQ ID NO:1). Preferably, the peptide comprises at least about six contiguous amino acids of the amino sequence set forth in SEQ ID) NO:1 or an analog or derivative thereof.

Examples of such peptides include PPLSQETFSDLWKLL (SEQ ID NO:1) or an analog or derivative thereof, PPLSQETFS (SEQ ID NO:2) or an analog or derivative thereof and ETFSDLWKLL (SEQ ID NO:3) or an analog or derivative thereof.

Thus, in accordance with the present invention, there are provided antibodies or immunologically active fragments thereof, which specifically bind PaCa-Ag1, and which antibodies are conjugated or linked to at least one of the peptides described above (SEQ ID NOs:1-3, or analogs or derivatives thereof). To improve transportation across a neoplastic cell membrane, a leader sequence is preferably positioned at the carboxyl terminal end of the peptide, analog, or derivative thereof. Preferably, the leader sequence comprises predominantly positively charged amino acid residues. Examples of leader sequences which may be used in accordance with the present invention include but are not limited to penetratin, Arg₈, TAT of HIV1, D-TAT, R-TAT, SV40-NLS, nucleoplasmin-NLS, HIV REV (34-50), FHV coat (35-49), BMV GAG (7-25), HTLV-II REX (4-16), CCMV GAG (7-25), P22N (14-30), Lambda N (1-22), Delta N (12-29), yeast PRP6, human U2AF, human C-FOS (139-164), human C-JUN (252-279), yeast GCN4, and p-vec. Preferably, the leader sequence is the penetratin sequence from antennapedia protein having the amino acid sequence KKWKMRRNQFWVKVQRG (SEQ ID NO:4).

In a preferred embodiment, there is provided a therapeutic composition for treating pancreatic cancer which comprises an antibody or binding portion thereof, having binding specificity to pancreatic carcinoma specific antigen 3C4-Ag (PaCa-Ag1) as described hereinabove, wherein the antibody or binding portion thereof is conjugated or linked to a peptide having the amino acid sequence set forth in SEQ ID NO:3, and wherein the carboxyl end if the peptide having the amino acid sequence as set forth in SEQ ID NO:3 is linked to a penetratin leader sequence having the amino acid sequence as set forth in SEQ ID NO:4.

Antibodies to 3C4-Ag and binding portions thereof may also be used in a drug/prodrug treatment regimen. For example, a first antibody or binding portion thereof according to the present invention is conjugated with a prodrug which is activated only when in close proximity with a prodrug activator. The prodrug activator is conjugated with a second antibody or binding portion thereof, preferably one which binds to pancreatic cancer cells or to other biological materials associated with pancreatic cancer cells such as another protein produced by the diseased pancreas cells. See e.g., Senter et al. (1988) Proc. Nat'l. Acad. Sci. (USA) 85:4842-46; and Blakely et al., (1996) Cancer Res. 56:3287-3292, both of which are incorporated by reference as if fully set forth.

Alternatively, the antibody or binding portion thereof may be coupled to a high energy radiation emitter, e.g., a radioisotope such as ¹³¹I, a γ emitter, which when localized at a tumor site, results in a killing of several cell diameters. See e.g., Order, in Monoclonal Antibodies or Cancer Detection and Therapy, Baldwin et al. (eds.) pp. 303-16, Academic Press, (1985). ⁶⁷Cu is also effective and may be attached to a subject antibody via an appropriate metal chelator which is bound to the antibody. Other suitable radioisotopes include α-emitters such as ²¹²Bi, ²¹³Bi, and ²¹¹At and β-emitters, such as ¹⁸⁶Re and ⁹⁰Y.

For therapeutic applications, chimeric (mouse-human) humanized monoclonal antibodies may be preferable to murine antibodies, since human subjects treated with mouse antibodies tend to generate antimouse antibodies. Antibodies may be “humanized” by designing and synthesizing composite variable regions which contain the amino acids of the mouse complementary determining regions (CDRs) integrated into the framework regions (FRs) of a human antibody variable region. Resultant antibodies retain the specificity and binding affinity of the original mouse antibody but are sufficiently human so that a patient's immune system will not recognize such antibodies as foreign. Techniques for humanizing mouse monoclonal antibodies include for example, those described in Vaswani et al., (1998) Ann. Allergy Asthma Immunol. 81:105-119 and U.S. Pat. No. 5,766,886 to Studnicka et al., the disclosures of which are incorporated by reference herein as if fully set forth.

In still another aspect of the invention, there is provided a eukaryotic expression vector comprising the exoplasmatic region of the human coxsackie adenoviral receptor and the variable region of an antibody specific to PaCa-Ag1 described hereinabove. The expression vector is useful for retargeting viral vectors such as Ad vectors in order to increase tissue specific infectivity. Immunological retargeting strategies based on the use of bispecific conjugates, or single chain antibodies displayed on a virus surface, i.e., a conjugate between an antibody directed against a component of a virus and a targeting antibody or ligand are known in the art. See, e.g., Douglas et al., 1996; Weitmann et al. 1992; and Hammond et al., 2001, the disclosures of which are incorporated by reference as if fully set forth.

The present invention further provides pharmaceutical compositions which may be used in the therapeutic methods described hereinabove. The pharmaceutical compositions comprise a pharmaceutically effective amount of an antibody or binding portion thereof which specifically recognizes and binds to 3C4-Ag or an immunologically active fragment thereof, and a pharmaceutically acceptable carrier. Examples of pharmaceutically acceptable carriers include sterile liquids such as water and oils, with or without the addition of a surfactant and other pharmaceutically and physiologically acceptable carrier, including adjuvants, excipients, or stabilizers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol are preferred liquid carriers, particularly for injectable solutions. Human serum albumin, ion exchangers, alumina, lecithin, buffer substances such as phosphates, glycine, sorbic acid, potassium sorbate, and salts or electrolytes such as protamine sulfate may also be used.

A subject pharmaceutical composition therefore comprises an antibody or binding portion thereof which specifically binds to 3C4-Ag or immunologically active fragment thereof, either unmodified, conjugated to a therapeutic agent (e.g., drug, toxin, enzyme, or second antibody as described hereinabove) or in a recombinant form such as a chimeric Ab. The pharmaceutical composition may additionally comprise other antibodies or conjugates for treating pancreatic cancer, such as e.g., an antibody cocktail.

Regardless of whether the antibodies or binding portions thereof of the present invention are used for treatment or in vivo detection of pancreatic cancer, they can be administered orally, parenterally, subcutaneously, intravenously, intralymphatic intramuscularly, intraperitoneally, by intranasal instillation, by intracavitary or intravesical instillation, intraarterially, intralesionally, or applied to tissue surfaces (including tumor surfaces or directly into a tumor) in the course of surgery. The antibodies of the present invention may be administered alone or with pharmaceutically or physiologically acceptable carriers, excipients, or stabilizers as described hereinabove. The subject antibodies may be in solid or liquid form such as tablets, capsules, powders, solutions, suspensions, emulsions, polymeric microcapsules or microvesicles, liposomes, and injectable or infusible solutions.

Effective modes of administration and dosage regimen for the antibody compositions of the present invention depend mostly upon the patient's age, weight, and progression of the disease. Dosages should therefore be tailored to the individual patient. Generally speaking, an effective does of the antibody compositions of the present invention may be in the range of from about 1 to about 5000 mg/m².

The following examples further illustrate the invention and are not meant to limit the scope thereof.

### Example 1

### Development of Cell Line BMRPA.430.NNK (BMRPA1.NNK) through Neoplastic Transformation of Pancreatic (Cell Line BMRPA.430

### Materials:

1640 RPMI medium, penicillin-streptomycin stock solution (10,000 U/10,000 mg/mL)(P/S), N-2-hydroxyethylpiperine-N′-2-ethanesulfonic acid (HEPES) buffer, 0.2% Trypsin with 2 mM Ethylene diamine tetraacetic acid (Trypsin-EDTA), and Trypan blue were all from GIBCO (New York). Fetal bovine serum (FBS) was from Atlanta Biologicals (Atlanta, Ga.). Dulbecco's Phosphate Buffered Saline without Ca²⁺ and Mg²⁺ (PBS), and all trace elements for the complete medium were purchased from Sigma Chemical Company (ST. Louis, Mis.). Tissue culture flasks (TCFs) were from Falcon-Becton Dickinson (Mountain View, Calif.), tissue culture dishes (TCDs) were obtained from Corning (Corning, N.Y.), 24-well tissue culture plates (TCP), and 96-well TCP were from Costar (Cambridge, Mass.). Filters (0.22, 0.45 μm) were from Nalgene (Rochester, N.Y.).

Preparation of Complex RPMI (cRPMI) Cell Culture Medium:

cRPMI was prepared with RPMI, glutamine (0.02M), HEPES-Buffer (0.02M), bovine insulin dissolved in acetic acid (0.02 mg/mL, acetic acid/L of medium), hydrocortisone (0.1 μg/mL), trace elements that included ZnSO₄ (5×10⁻⁷M), NiSO₄6H₂O (5×10⁻¹⁰ M), CuSO₄ (10⁻⁸M), FeSO₄ (10⁻⁶M), MnSO₄ (10−9M), (NH₄)₆Mn₇O₂₄ (10⁻⁷M), Na₁SeO₃ 3, (0.5 mg/L medium), SnCl₂ 2H₂O (5×10⁻¹⁰M) and carbamyl choline (10⁻⁵M), and the pH was adjusted to 7.3. The medium was sterile filtered.

### Cells and Culture:

BMRPA.430 (BMRPA1) is a spontaneously immortalized cell line established from normal rat pancreas (Bat et at, 1994). TUC3 (BMRPA1.K-ras^{Val12}) are BMRPA1 cells transformed by transfection with a plasmid containing activated human K-ras with oncogenic mutation at codon 12 (Gly->Val) (Dr. M. Perucho, California Institute for Biological Research, La Jolla). All cell lines are maintained routinely in cRPMI (10% FBS) in a 95% air-5% CO₂ incubator (Forma Scientific) at 37° C. The cells are passaged by trypsin-EDTA. Cells are stored frozen in a mixture made of 50% spent medium and 50%, freezing medium containing fresh cRPMI with 10% FBS and 10% DMSO. Cell viability was assessed by trypan blue exclusion.

### NNK Exposures:

All preparations of the carcinogen-containing media were made in a separate laboratory within a NCI-designed and certified chemical hood using prescribed protective measures. 4-(N-nitrosmethylamino)-1-(3-pyridyl)-1-butanone (NNK, American Health Foundation, N.Y.) was prepared as a stock solution of 10 mg NNK in PBS and added to FBS-free cRPMI to make final concentrations of 100, 50, 10, 5, and 1 μg/mil. BMRPA1 cells at passage 36 (p36) were seeded at 10⁵/60 mm TCDs and allowed to grow for 6 d. At this time the medium was removed, and the cells were washed 2× with prewarmed (37° C.), FBS-free cRPMI before they were treated with FBS-free cRPMI (4 ml/TCD) containing the different concentrations of NNK. A 6th set of TCDs containing BMRPA1 cells was incubated in FBS-free cRPMI without NNK and was used as controls. The eight TCDs used for each of the six sets of different culture conditions were returned to the 37° C. and 95% air-5% CO₂ incubator. After 16 h, the NNK-containing medium was removed from all TCDs and the cells were washed 3× with PBS followed by addition of fresh cRPMI-10% FBS (4 ml/TCD), and the incubation continued. Control cultures without NNK were processed in parallel. The cells were fed every 2 d by replacing ½ of the spent medium with fresh cRPMI-10% FBS. At full confluency the cells were collected from all TCDs, the cells in each group were pooled, and passaged at 2×10⁴ into fresh TCDs.

### Isolation of Colonies:

To facilitate the picking of cells from individual colonies of transformed cells, cell cultures containing colonies were reseeded at 10⁵ cells/10 mm TCDs, and grown for 7 d. The narrow ends of sterile Pasteur pipettes were flamed, rapidly stretched and broken at their thinnest point to create a finely drown-out glass needle narrow enough to pick up only the core of a cell-rich colony. Only the NNK treated cells contained cell-rich, ball-like colonies. The center cores of 8 prominent colonies were picked, and each core consisting of ˜80-200 tightly packed cells was placed into a separate well each of a 24-well dish. The cells of 4 colonies thus transferred survived and were expanded.

### Cell Growth Assays:

To measure cell growth at 10% FBS, cells were seeded at 5×10⁴ cell/60 mm TCD containing 4 ml of cRPMI-10% FBS. Every 3 d, triplicate TCDs were removed for each cell line under study, the cells were released with trypsin-EDTA, and counted in the presence of trypan blue. To assess the effect of cRPMI containing reduced FBS concentrations on cell growth, equal numbers (1.5×10⁴ cell/ml/well) of NNK-treated and untreated BMRPA1 cells were seeded in triplicate wells of 24 well TCDs. The cells were allowed to adhere overnight in cRPMI 10% FBS, washed with PBS, and reincubated with cRPMI containing the indicated % FBS. Cell growth was evaluated by a modification of the crystal violet relative proliferation assay (Serrano, 1997). Briefly, the cells were washed with PBS, fixed in 10% buffered formalin followed by rinsing with distilled water. The cells were then stained with 0.1% Crystal Violet for 30 min at room temperature (RT), washed with dH₂O, and dried. The cell-associated dye was extracted with 1 ml 10% acetic acid, aliquots were diluted 1:2 with dH₂O, and transferred to 96-well microtiter plates for OD₆₀₀ₙₘ measurements. The cell growth was calculated relative to the OD₆₀₀ₙₘ values read at 24 h.

### BrdU Incorporation:

Cells (5×10⁴) were plated in 60 mm TCD, and allowed to grow in cRPMI-10% FBS. Three days later, fresh medium with BrdU (10 uM) was added for 3 h, the cells were washed, released with Trypsin-EDTA, and the incorporated BrdU was detected with an FITC conjugated anti-BrdU antibody (Becton Dickinson) by FACS analysis as suggested by manufacturer (Becton Dickinson). Briefly, 10⁶ trypsin-EDTA released cells were washed twice in PBS-1% BSA, fixed in 70% ethanol for 30 min, and resuspended in RNAase A (0.1 mg/mL) for 30 min at 37° C. After washing the cells, their DNA was denatured with 2N HCl/Triton X-100 for 30 min, and neutralized with 0.1 M Na₂B₄O₇.10H₂O, pH 8.5. The cells were then washed in PBS-1% BSA with 0.5% Tween 20, and resuspended in 50 uL of 0.5% Tween in PBS-1% BSA solution with 20 uL of FITC-AntiBrdU antibody. After 45 min at 37° C., the cells were washed, resuspended in 1 mL of Na Citrate buffer containing Propidium Iodide (0.005 mg/mL) and RNAase A (0.1 mg/mL). Fluorescent activated cell sorting or flow cytometry (FACS) analysis to detect the incorporated BrdU and PI staining was performed by using a FACScan analyzer from Becton Dickinson Co. equipped with an Argon ion laser using excitation wavelength of 488 nm. Data analysis was performed using the LYSYS II program.

Independent samples t-test was used to show statistically significant (p<0.05) differences in the percentage of the untransformed and transformed cells that incorporate BrdU. The DNA index was calculated as previously described (Barlogie et al., 1983; Alanen et al., 1990) from the DNA histogram as the ratio of the P1 staining measurement for the GO/GI peak in the transformed cells examined divided by the PI staining measurement for the G0/G1 peak in the untransformed BMMRPA1 cells.

### Anchorage Independent Growth:

Aliquots of 4 ml of 0.5% agar-medium mixture (agar was autoclaved in 64 mL H₂O, cooled in a water bath to 50° C., and added to 15 mL 5× cRPMI, 19 mL FBS and 1 mL P/S) were poured into 25 cm² TCFs and allowed to harden overnight at 4° C. Prior to plating the cells, the flasks were placed in the COT-Air incubator for up to 5 h at 37° C. to facilitate equilibration of pH and temperature. Cells were collected by Trypsin-EDTA, 0.1 mL, of cell suspension (40000/mL cells in cRPMI) was dispersed carefully over the agar surface of each flask and the cultures were returned to the 37° C. incubator with 95% O₂-5% CO₂. After 24 h, the agar-coated TCFs were inverted to allow drainage of excess medium. The cultures were examined microscopically after 9 d and 14 d for growth of colonies using a Zeiss inverted microscope.

Tumorigenicity in Nu/Nu mice:

Nu/Nu mice (7 wks of age) were obtained from Harlan laboratories (Indianapolis, Ind.). The cells used for injection were released by Trypsin-EDTA, washed in cRPMI, and resuspended in PBS at 10⁸ cells/mL. Each mouse tested was injected subcutaneously (s.c.) with 0.1 ml of this cell suspension. The animals were inspected for tumor development daily during the first 4 weeks, and thereafter at weekly intervals. Small pieces of the tumors (1-2 mm.) were cut from the core of the tumors and placed in 4% pafaformaldehyde overnight at 4 C. The tissue was then washed in PBS, and placed in 30% sucrose for another 24 h. Sections of tumor tissue frozen in Lipshaw embedding matrix (Pittsburgh, Pa.) were made with a Jung cryostat (Leica), placed on gelatin coated slides, and stored at −20 C. H&E staining was done according to standard procedures.

Establishment of the TUNNK Cell Line from Excised Nu/Nu Mice Tumors:

Isolation of cells from tumors that grew from the BMRPA1.NNK cells that had been transplanted subcutaneously into Nu/Nu mice was done similar to the method described by Amsterdam, A. and Jamieson, J. D., 1974, J. Cell Biol. 63:1037-1056, with several procedural changes. The tumor-bearing Nu/Nu mice were sacrificed by CO₂ asphyxiation, placed on an ice-cooled bed, the skin over the tumor opened and the tumor rapidly removed surgically and sterilely, and placed into L-15 medium (GIBCO, Grand Island, N.Y.) on ice for immediate processing. While still in ice-cold L-15 medium, the tissue was minced into small pieces, followed by 2 cycles of enzymatic digestion and mechanical disruption. The digestion mixture in L-15 medium consisted of collagenase (1.5 mg/ml) (136 U/mg; Worthington Biochem. Corp.), Soybean trypsin inhibitor (SBTI) (0.2 mg/ml) (Sigma Chem. Comp.), and bovine serum albumin (BSA; crystallized) (2 mg/ml) (Sigma). After the first digestion cycle (25 min. 37° C.), the cells and tissue fragments were pelleted at 250×g, and washed once in ice-cold Ca⁺⁺ and Mg⁺⁺-free phosphate buffered saline (PD) containing SBTI (0.2 mg/ml), BSA (2 mg/ml), EDTA (0.002 M) and HEPES (0.02 M) (Boehringer Mannheim Biochem., Indianapolis) (S-Buffer). The cells were pelleted again, resuspended in the digestion mixture, and subjected to the second digestion cycle (50 min, 37° C.). While still in the digestion mixture, the remaining cell clumps were broken apart by repeated pipetting of the cell suspension using pipettes and syringes with needles of decreasing sizes. The cell suspension was then sheared sequentially through sterile 200μ-mesh and 20μ-mesh nylon Nytex grids (Tetko Inc., Elmsford, N.Y.), washed in S-Buffer and resuspended in 2-3 ml L-15 medium, centrifuged at 50×g for 5 min at 4° C. The cell pellet was collected, washed in PBS, and resuspended in cRPMI, A sample of the fraction was processed for viable cell counting by Trypan blue (Fisher Sci.) exclusion (Michl J. et al., 1976, J. Exp. Med. 144(6), 1484-93) and for cytochemical analysis. Cells were seeded and grown in cRPMI at 10⁵ cells/35 mm well of a 6-well TCD.

### Photomicroscopy:

All observations and photography of cell cultures were done on a Leitz Inverted Microscope equipped with phase optics and a Leitz camera. Observations were recorded on TMX ASA100 Black and White film.

### Example 2

### Results

### Effects of NNK on BMRPA1 Morphology:

Repeated exposures to NNK, and other nitrosamines have been observed to induce both cytotoxic and neoplastic morphological alterations in a variety of rodent and human in vitro experimental models of pancreatic cancer (Jones, 1981, Parsa, 1985, Curphey, 1987, Baskaran et al. 1994). With the purpose of determining whether such changes are induced by a single exposure to NNK and at relatively small NNK concentrations, BMRPA1 cells were exposed for one 16 hour period to serum free medium containing 100, 50, 10, 5, and 1 μg NNK/mL. As observed in previous studies with pancreatic cells, the larger concentrations of NNK resulted in cylotoxic changes consisting of poorly attached, degenerating, dying cells, and slowed cell growth, while such changes were observed considerably less in cells exposed to 5, and 1 μg NNK/mL. The degenerative changes of the treatment with 100, 50, 10 μg NNK/ml were followed within a week by the appearance of phenotypical changes indicative of neoplastic transformation such as spindle morphology and focal overcrowding. BMRPA1 cells treated with NNK at 1 μg/ml also displayed phenotypical changes characteristic of neoplastic transformation but at a slower rate, over several weeks. As suggested for other mutagens (Srivastava and Old, 1988), the changes observed at lower doses might be more likely to reflect specific, preferential molecular sites of NNK-induced lesions at doses closer to those encountered in the human environment. Furthermore, the gradual pace of these changes at 1 μg/mL allows a passage by passage study of both early and late events in the process of NNK-induced transformation. Thus, the results presented below were obtained with BMRPA1 cells exposed once for 16 h to 1 μg NNK/mL FBS-free medium.

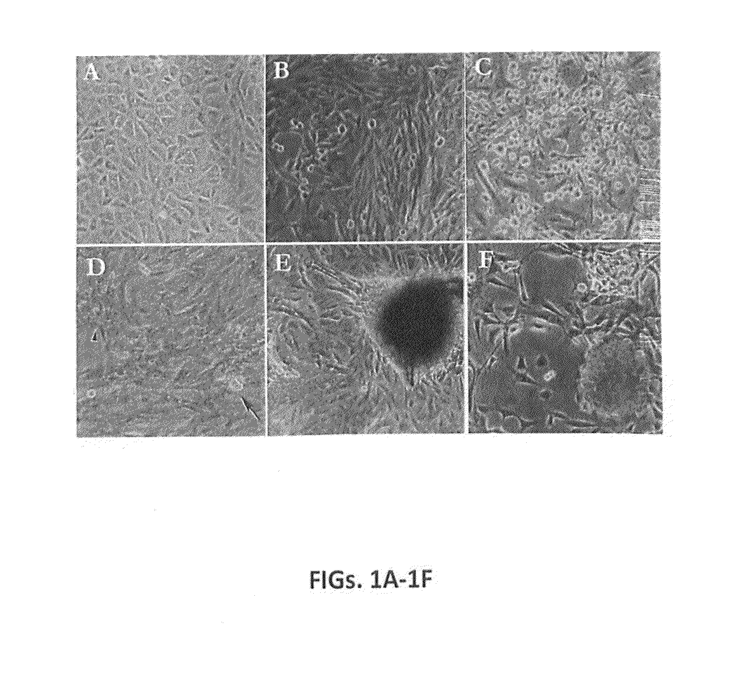

BMRPA1 cells grown continuously in culture for 35 passages were organized into a monolayer, cobblestone-like pattern typical of untransformed, contact inhibited epithelial cells (FIG. 1A). Two weeks after exposure to 1 μg NNK/ml, the BMRPA1 cells exhibited minute morphological changes: cells in a few discrete areas started losing their polygonal shape, and islands of cells consisting of spindle-shaped cells with less cytoplasm and darker nuclei started forming (FIG. 1B, passage 2 or p2). Beginning with p6 an increasing number of round cells on top and within the strands of densely packed spindle cells were observable (p6-8), suggesting loss of contact inhibition (FIG. 1C).

Island-like areas of crowded cells (foci) became prominent by p7 (FIG. 1D, arrow tip), and ball-like aggregations of cells began to form on the top of these foci as colonies (p7-11). The first clearly distinguishable colonies were seen at p8-9, about 3 months after NNK exposure. Initially the colonies were small (FIG. 1D, arrow) and only few, but they were present in all 6 TCFs in which the NNK-treated BMRPA1 cells were passaged. The colonies continued to grow horizontally and vertically as compact masses (FIG. 1E) with much reduced adhesiveness, e.g., crowded cells could be easily separated by trypsinization and repeated pipetting, indicating that such cultures likely comprise neoplastic cells. The rapid disruption by trypsinization of such colonies is in direct contrast to untransformed BMRP430 (BMRPA1) cells. The control BMRPA1 cells that had been continuously cultured in parallel after 16 h exposure to FBS-free cRPMI without NNK did not show any changes and were indistinguishable from the original monolayer of BMRPA1 cells.

To facilitate the study of phenotypical and molecular characteristics of colony-forming cells, the cores of several colonies were isolated with a finely drown out glass needle, and each isolate of 80-200 cells was grown separately as cell lines referred to as “cloned BMRPA1.NNK”. The isolated cells displayed a spindle to triangular shape and were often multi-nucleated with different sized nuclei containing one or more prominent nucleoli. When reseeded in new flasks, these cells maintained the ability to form foci and colonies (FIG. 1F). Interestingly, the NNK-induced phenotypic changes seen in the NNK-transformed BMRPA1 are similar to but less pronounced than those observed during the transformation of BMRPA1 by human oncogenic K-ras^{val12} The NNK-induced basophilic foci that can be easily observed macroscopically and microscopically after H&E staining are also similar to those formed by BMRPA1 cells transformed by transfection with oncogenic K-ras^{val12}. In contrast, neither foci nor colonies were formed during the growth of untreated BMRPA1 cells. The morphological changes induced by NNK in BMRPA1 cells are also similar to well-established characteristics of other transformed cells cultured in vitro: spindly and triangular cell shape at low cell density, rounded with halo-like appearance at high cell density, and loss of contact inhibition as indicated by growth in foci and on top of their neighboring cells (Chung, 1986).

NNK-Induced Hyperproliferation:

The long-term, permanent effects of NNK on the proliferation of BMRPA1 cells was initially assessed by comparing the cell growth of NNK-treated and untreated cells cultured in complex medium (cRPMI) supplemented with 10% FBS. The BMRPA1, uncloned NNK-treated BMRPA1 cells, and “cloned” BMRPA.1NNK cells, i.e., isolated cells produced as described above, this example, were seeded at equal density in TCDs. At predetermined days the cells in TCDs were released by Trypsin-EDTA, collected, and counted in the presence of trypan blue. Untreated BMRPA1 cells at passage 46 (p46) reached a plateau around day 9 indicative of contact inhibited growth. In contrast, the NNK-treated cells grown in parallel for eleven passages after the NNK, treatment showed faster growth during the first 9 d, and later the growth slowed down possibly due the continued presence of untransformed BMRPA1 cells that were unaffected by NNK. The cloned BMRPA.1NNK, cells isolated from the core of the NNK-induced colonies (FIG. 1F) continued to grow unimpeded throughout the 12 days of culture at a considerably faster rate than the untreated BMRPA1 cells resulting in very dense overcrowding.

Since the cell growth curves were able to reveal significant growth differences between the NNK-treated and untreated BMRPA1 cells only at high cell densities where contact inhibited growth and cell death might contribute significantly to the observed cell growth, the increased intrinsic capacity of the NNK-treated cells to proliferate at low cell density was further assessed by measuring the ability of these cells to incorporate BrdU. The measurement of BrdU incorporation in RNAase treated cells is routinely used to assess DNA synthesis during the S phase of proliferating cells (Alberts B., Johnson, A., Lewis, J., Raff, M., Roberts, K., Walter, P., 2002, Molecular Biology of the Cell. Garland Science, Taylor and Francis, 4th ed., NY). The results obtained by FACS analysis of the BrdU incorporation in the untransformed BMRPA1.p58, transformed uncloned BMRPA.NNK.p11, and transformed cloned BMRPA.NNK.p23 cells offer further evidence that the NNK treatment resulted in permanent hyperproliferative changes in BMRPA1. These observations provide experimental evidence that NNK is able to transform BMRPA1 cells by inducing both a focal loss of contact inhibition and hyperproliferation.

### Effect of Serum Deprivation on Untransformed and NNK-Transformed BMRPA1 Cells:

One frequently cited characteristic of transformed cells is their selective growth advantage at low concentrations of growth factors and serum, conditions that poorly support the growth of primary and untransformed cells (Chung, 1986; Friess, et al., 1996; Katz and McCormick 1997). To establish the serum dependency of the untransformed and NNK-transformed BMRPA1, cells were transferred into cRPMI medium supplemented with 1%, 5%, and 10% FBS, seeded at equal cell numbers into the wells of 24-well TCPs, and grown for 12 days. A crystal violet assay was used to assess the relative cell growth (Serrano, 1997). This assay provides a significant advantage over the counting of cells released by Trypsin-EDTA because it eliminates the loss of cells (incomplete release and cell death) that occurs due to strong cell adhesion to TCDs at low serum concentrations.

It was found that transformed BMRPA.1NNK cells have a selective growth advantage over untreated cells at all the FBS concentrations examined. Even in cRPMI medium containing 1% PBS the NNK-transformed cells grow better than untreated BMRPA1 cells cultured in cRPMI with 10%. The observed ability of BMRPA1.NNK cells to sustain cell growth in severely serum-deprived conditions provides further support for the transformation of BMRPA1 cells by exposure to NNK.

### Anchorage-Independent Cell Growth:

The malignant transformation of many cells has been shown to result in a newly acquired capability to grow on agar, under anchorage independent conditions (Chung, 1986). The ability of the cloned BMRPA.1NNK and untreated BMRPA1 cells to grow on agar was examined by dispersing cells at low density onto soft agar (see Example 1). The ability of these cells to form colonies over a 14 d period is presented in Table 1.

TABLE 1
Anchorage independent colony formation on agar by control BMRPA1
and NNK-treated BMRPA1 cells

| | Days after | # of colonies* formed | | |
|---|---|---|---|---|
| Cells | seeding | <50 cells | >50 cells | Total |
| BMRPA1 | 9 | 0 | 0 | 0 |
| | 14 | 0 | 0 | 0 |
| BMRPA1.NNK | 9 | 14 | 15.8 ± 2.5 | 17.3 ± 5.2 |

*using an ocular counting grid the colonies were counted in a series of 30 sequential 1 mm² fields Average counts of colonies from 5 TCFs ± SEM are presented.

Confirming previous observations (Bao et al., 1994), the BMRPA1 cells were unable to grow on agar and died. In contrast, BMRPA1.NNK cells showed a strong capacity to grow and form colonies. In fact, about 1 in 4 BMRPA1.NNK cells seeded formed colonies larger than 50 cells. The growth on agar is indicative of neoplastic transformation

### Tumorigenicity in Nu/Nu Mice:

Cells growing on agar often have the ability to grow as tumors in Nu/Nu mice (Shin et al., 1975; Colburn et al., 1978). The ability of cells to grow in Nu/Nu mice as tumors is believed to be a strong indication of malignant transformation (Chung, 1986). Consequently, 10⁷ cloned, live BMRPA1.NNK cells were injected subcutaneously (s.c.) in the posterior flank region of Nu/Nu mice. Another group of mice was injected s.c. under similar conditions with untransformed BMRPA1 cells. A third group of Nu/Nu mice was injected with BMRPA1.K-ras^{val12} cells for positive control purposes, since these cells have been previously shown to form tumors in Nu/Nu mice.

TABLE 2
Tumorigenieity of BMRPA1.NNK cells in Nu/Nu mice.

| | # of mice with | # of mice with |
|---|---|---|
| | tumor/# of | metastasis/# of |
| Cells | mice tested | mice tested |
| BMRPA1 | 0/5 | 0/5 |
| BMRPA1.NNK | 3/6 | 1/6 |
| BMRPA1.K-ras^{val12} | 5/5 | 1/5 |

BMRPA1 cells were unable to form tumors in the 5 Nu/Nu mice injected, while BMRPA1.K-ras^{val12} formed rapidly growing nodules (<0.5 cm) that became tumors (>1 cm) within 4 wks after inoculation. Distinctly different was the course of tumor formation in the Nu/Nu mice injected with cloned BMRPA1.NNK cells. Within a week after injection with cloned BMRPA1.NNK cells, nodules of 2-3 mm formed at the injection site of all six mice. The nodules disappeared in 3 of the animals within 2 months. Nevertheless, after a period of dormancy of up to 4 months, the nodules in the remaining 3 animals evolved within the next 12-16 weeks into tumors of more than 1 cm in diameter. One of these mice carrying a large tumor mass further developed ascites indicating the presence of metastatic tumor cells.

A cell line named TUNNK, was established from one of the tumors growing in BMPRA1.NNK injected Nu/Nu mice by a method combining mechanical disruption and collagenase digestion. TUNNK has transformed morphological features similar to the cloned BMRPA1.NNK cells injected into the Nu/Nu mouse. So far, the only prominent distinguishing phenotypical characteristic between the two is a predisposition of TUNNK to float in vitro as cell aggregates, suggesting that significant changes in the adhesion properties of the cells took place during the selective growth process in vivo.

### Example 3

### Tolerance-Induced Targeted Antibody Production (TITAP)

### Materials and Methods:

Materials:

RPMI 1640, DMEM containing 5.5 mM glucose (DMEM-G+), penicillin-streptomycin, HEPES buffer, 0.2% trypsin with 2 mM EDTA, Bovine serum albumin (BSA). Goat serum, and Trypan blue were from GIBCO (New York). Fetal bovine serum (FBS) was from Atlanta Biologicals (Atlanta, Ga.). Hypoxanthine (H), Aminopterin (A), and Thymidine (T) for selective HAT and HT media and PEG 1500 were purchased from Boehringer Mannheim (Germany). Diaminobenzidine (DAB) was from BioGenex (Dublin, Calif.). PBS and Horseradish peroxidase labeled goat anti-Mouse IgG [F(ab′)₂ HRP-GαM IgG] were obtained from Cappel Laboratories (Cochranville, Pa.). Aprotinin, pepstatin, PMSF, sodium deoxycholate, iodoacetamide, paraformaldehyde. Triton X-100, Trizma base, OPD, HRP-GαM IgG, and all trace elements for the complete medium were purchased from Sigma (ST. Louis, Mo.). Ammonium persulfate, Sodium Dodecyl Sulfate (SDS), Dithiothreitol (DTT), urea, CHAPS, low molecular weight markers, and prestained (Kaleidoscope) markers were obtained from BIORAD (Richmond, Calif.). The enhanced chemiluminescent (ECL) kit was from Amersham (Arlington Heights, Ill.). Mercaptoethanol (2-ME) and film was from Eastman Kodak (Rochester. N.Y.). Tissue culture flasks (TCF) were from Falcon (Mountain View, Calif.), tissue culture dishes (TCDs) from Corning (Corning, N.Y.), 24-well ITC plates (TCPs) and 96-well TCPs were from Costar (Cambridge, Mass.). Tissue culture chambers/slides (8 chambers each) were from Miles (Naperville, Ill.).

Cells and Culture:

All rat pancreatic cell lines were grown in cRPMI containing 10% FBS. The other cell lines were obtained from the American Tissue Culture Collection (ATCC), except for the rat capillary endothelial cells (E49) which were from Dr. M. DelPiano (Max Planck Institute, Dortmund, Germany). White blood cells were from healthy volunteer donors, and human pancreatic tissues (unmatched transplantation tissues) were provided by Dr. Sommers from the Organ Transplantation Division at Downstate Medical Center. Cell viability was assessed by trypan blue exclusion.

Immunosubtractive Hyperimmunization Protocol (ISHIP):

The ISHIP protocol is described in detail in copending application Ser. No. 60/443,703, the disclosure of which is incorporated by reference as if fully set forth. A mixture of live (10⁶) and paraformaldehyde fixed and washed (10⁶) cells was used for each immunization intraperitoneally (ip). Six female Balb/c mice (age˜12 wks) were used: two mice were injected 4× during standard immunizations with BMRPA1 cells. The other four mice were similarly injected 3× with BMRPA1 cells, and 5 h after the last booster injection they were injected ip for the next 5 d with 60 μg cyclophosphamide/day/g of body weight. Two of these immunosuppressed mice were re-injected with BMRPA1 cells after the last Cy injection. The other two immunosuppressed mice were injected weekly three more times with transformed BMRPA1.NNK cells, and a week later the mice were hyperimmunized with 5 additional injections in the 7 days preceding fusion (ISHIP mice). Sera were obtained from all mice within a week after the indicated number of immunizations.

Hybridoma and mAb purification:

Hybridomas were obtained as previously described (Kohler and Milstein, 1975; Pytowski et al., 1988) by fusion of P3U1 myeloma cells with the splenocytes from the most immunosuppressed ISHIP mouse. Hybridoma cells were cultured in 288 wells of 24-well TCPs. The hybridomas were initially grown in HAT DMEM-G+(20% FBS) medium for 10 d, followed by growth in HT containing medium for 8 d, and then in DMEM-G+ (20% FBS). Hybridoma supernatants were tested 3× by Cell-Enzyme ImmunoAssay (Cell-EIA) starting 3 weeks after fusion for the presence of specific reactivities by Cell-EIA before the selection of specific mAbs for further analysis by immunofluorescence microscopy and immunohistochemistry was made.

### Example 4

### Detection of Antigenic Differences Between NNK-Transformed and Untransformed BMRPA1 Cells

Hybridoma supernatants collected from 288 wells were tested by Cell-Enzyme ImmunoAssay (Cell-EIA) for the presence of IgG antibodies reactive with dried NNK-transformed and untransformed BMRPA1 cells. BMRPA1 and BMRPA1.NNK cells were seeded in TCPs (96-wells) at 3×10⁴/well with 0.1 mL cRPMI-10% FBS. The cells were allowed to adhere for 24 h, air dried, and stored under vacuum at RT. The cells were then rehydrated with PBS-1%, BSA, followed by addition of either hybridoma supernatants or two fold serial dilutions of mouse sera to each well for 45 min at room temperature (RT). After washing with PBS-BSA, HRP-GαMIgG (1:100 in PBS-1% BSA) was added to each well fir 45 min at RT. The unbound antibodies were then washed away, and OPD substrate was added for 45 min at RT. The substrate color development was assessed at OD₄₉₀ₙₘ with a microplate reader (Bio-Rad 3550). For hybridoma supernatants, an OD₄₉₀ₙₘ value greater than 0.20 (5× the negative control OD₄₉₀ₙₘ value obtained with unreactive serum) was considered positive. Evaluation on days 18 to 21 after fusion established that 265 (92%) of the 288 wells examined contained one or more growing hybridomas. By Cell-EIA, supernatants from 73 (or 23.5%) of the wells contained antibodies that reacted with transformed BMRPA1.NNK cells. In contrast, only 47 (or 16.3%) supernatants reacted with BMRPA1 cells, indicating that BMRPA1.NNK cells express antigens which are not expressed by the untransformed BMRPA1 cells. Moreover, all 47 hybridoma supernatants reactive with BMRPA1 cells exhibited cross reactivity with transformed BMRPA1.NNK cells.

### Example 5

### Immunoreactivity of Selected Hybridoma Supernatants with Intact Untransformed and Transformed BMRPA1 Cells

As the Cell-EIA testing was performed on dried, broken cells, the antibodies in the supernatants could access and bind both intracellular and plasma membrane Ags. To obtain initial information regarding the cellular location of the recognized Ags, 5 hybridoma supernatants were initially selected for further testing by Indirect Immunofluorescence Assay (IFA) on intact cells because by Cell-EIA these supernatants consistently showed promising strong reactivity either with only BMRPA1.NNK cells (supernatants 3A2; 3C4; 3D4), or with both BMRPA1.NNK and BMRPA1 cells (supernatants 4AB1; 2B5). Supernatants 3C4, 4AB1, and 2B5 stained the cell surface of intact cells in agreement with the Cell-EIA results.

Cells were released by incubation with 0.02 M EDTA in PBS, washed with PBS-1% BSA, and processed live at ice cold temperature for immunofluorescence analysis. The cells were incubated for 1 h in suspension with hybridoma supernatants or sera, washed (3×) in PBS-1% BSA, and exposed to FITC-Gα M IgG diluted 1:40 in PBS-1% BSA. After 45 min. unbound antibodies were washed away, and the cells were examined by epifluorescence microscopy.

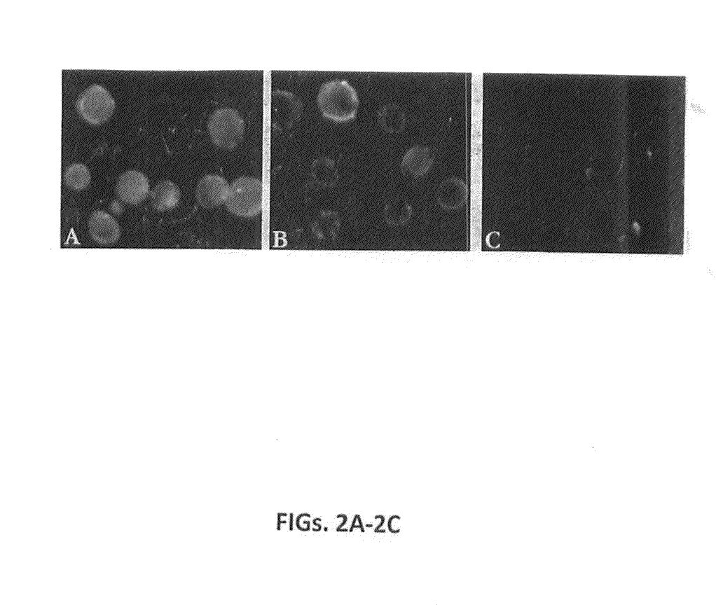

Remarkably, 3C4 stained BMRPA1.NNK (FIG. 2B) and BMRPA1.K-ras^{val12} cells (see copending provisional patent application, Ser. No. 60/443,703) in a ring-like pattern, but did not stain the cell surface of untransformed BMRPA1 cells (FIG. 2C), indicating the presence of the 3C4-Ag on the surface membrane of only transformed cells.

### Example 6

### Immunoperoxidase Staining of Permeabilized Cells and Tissue Sections

Preparation of Cells and Tissues:

Transformed and untransformed BMRPA1 cells were seeded at 1×10 cells/0.3 mL cRPMI/chamber in Tissue Culture Chambers. Two days later, the cells were fixed in 4% paraformaldehyde in PBS overnight at 4° C. The cells were then washed twice with PBS-1% BSA and used for immunocytochemical staining. Pancreatic tissue for immunohistochemical staining was prepared from adult rats perfused with 4% paraformaldehyde in 0.1M phosphate butter, pH 7.2. The fixed pancreas was removed from the fixed rat and stored overnight in 4% buffered paraformaldehyde at 4° C. The pancreas was then washed and placed in 30% sucrose overnight. Frozen tissue sections (10 μm) were made with a Jung cryostat (Leica), placed on gelatin-coated glass slides, stored at −20° C. The cell lines or tissue sections were then post-fixed for 1 min in 4% buttered parafomaldehyde, washed in Tris buffer (TrisB) (0.1 M, pH 7.6), and placed in Triton X-100 (0.25% in TrisB) for 15 min at RT. Immunohistochemistry was then performed as previously described (Guz et al., 1995).

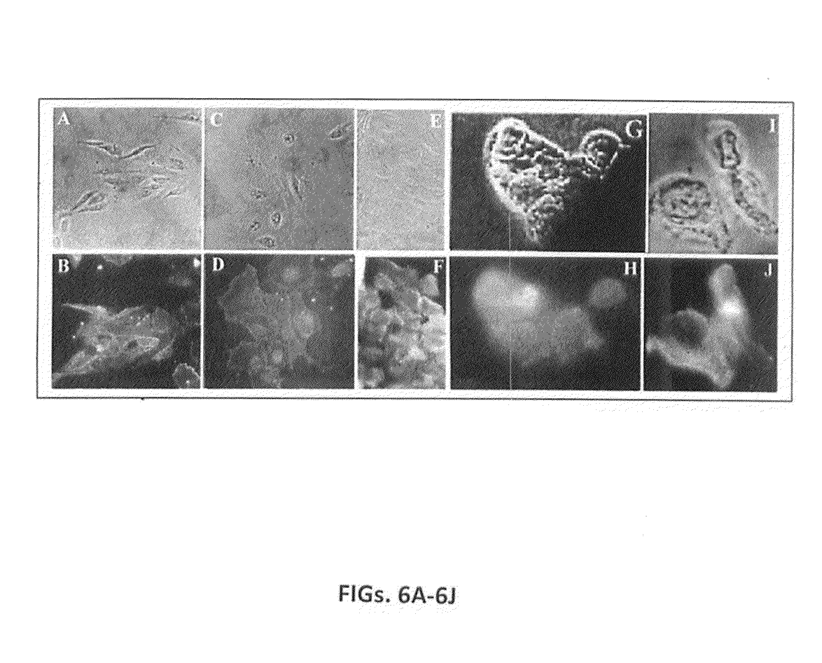

If staining with mAb3C4 of live rodent and human PaCa cells localized the 3C4-Ag to the plasma membrane of the intact cells (FIGS. 6A through 6J). The 3C4 staining detected by IFA and FACS (Example 7) was totally abolished when trypsin/EDTA instead of only EDTA was used to release the cells, indicating that the 3C4 Ag is a trypsin-sensitive protein found on the outer membrane of transformed BMRPA1 cells.

### Example 7

### Fluorescence Activated Cell Sorting Analysis (FACS) of Transformed and Untransformed Rodent and Human Pancreatic Carcinoma Cells

Live cells were placed on ice and reacted sequentially with mAb3C4 and Fluorescein Isothiocyanate (FITC-) labeled rabbit-αM IgG (FITC-RαM IgG), fixed overnight in 2% buffered paraformaldehyde, washed and analyzed on a BD FACS IV analyzer.

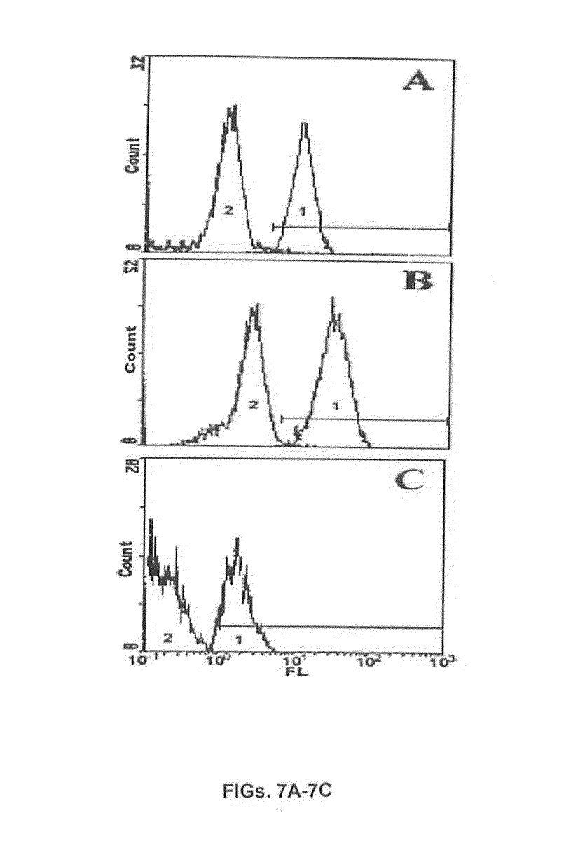

FACS analysis of stained BMRPA1.TUC3 cells provided a semi quantitative assessment of the presence of the antigen on the surface of the cells and confirmed fluorescence on >99% of the cells, indicating that >99% of the cells in each of the PaCa cell population expressed the 3C4-Ag. These results are shown in the fluorescence intensity graphs of FIG. 7.

### Example 8

### Purification of mAb3C4

Mice were injected with 3C4 hybridoma cells (10⁷/mouse). Ascites were collected and mAb3C4 IgG1 was purified from the ascites using G-protein affinity beads. Protein G beads were incubated under constant rotation overnight at 4° C. with ascites extracted from mice injected intraperitoneally (i.p.) with mAb3C4-producing hybridoma cells. The protein G beads were then centrifuged, the supernatant was removed, and the heads washed sequentially with Buffer A (10 mM Tris, 2 mM EDTA, 100 mM NaCl, pH 7.5). Buffer B (10 mM Tris HCl, 200 mM NaCl, 2 mM of EDTA, 0.2% Triton X-100, 0.25 mM PMSF pH 7.5), and Buffer C (10 mM Tris HCl, 0.25 mM PMSF pH 7.5) to remove non-specifically adsorbed proteins. Bound mAb3C4 was eluted from the heads with two bead volumes of elution buffer (0.1 M Glycine pH 2.7) followed each time by neutralization of the eluate with 1M Tris-HCl, pH 9.0 after its separation from the beads by brief centrifugation.

The purification of the mAb3C4 IgG was confirmed by SDS-PAGE and Immunoblotting (IB).

SDS PAGE and Immunoblotting (IB) of mAb3C4:

The mAb3C4 eluted and separated from the protein G-beads column were subjected to SDS PAGE under reducing and non-reducing conditions and immunoblotting (IB). mAb3C4 samples as well as other samples described below, were mixed with equal volumes of non-reducing sample buffer (125 mM Tris-HCl, 2% SDS, 0.1% bromophenol blue, 20% v/v glycerol, pH 6.8) and reducing sample buffer (125 mM Tris-HCl, 2% (v/v) 2-mercaptoethanol, 2% SDS, 0.1% bromophenol blue, 20% v/v glycerol, pH 6.8) The proteins from each sample (20 μg/well) were separated by SDS-PAGE as previously described (Laemmli, 1970), and electrotransferred onto nitrocellulose membrane. Gel lanes were loaded as follows:

| Lane | Sample |
|---|---|
| 1 = | Hybridoma injected mouse ascites |
| 2 = | Low pH buffer elution of proteins from protein-G beads incubated |
| | with ascites |
| 3 = | Proteins of Lane 2 after Reduction |
| 1B = | IB of Lane 1 |
| 2B = | IB of Lane 2 |

After the membrane was incubated with 5% (w/v) dry milk in TBS-T for 1 h, the HRP-GαM IgG antibody was used as suggested by the manufacturer (ECL kit, Amersham). The presence of the mAb3C4 protein by ECL in each of the samples tested was detected by exposure to X-OMAT film (Kodak).

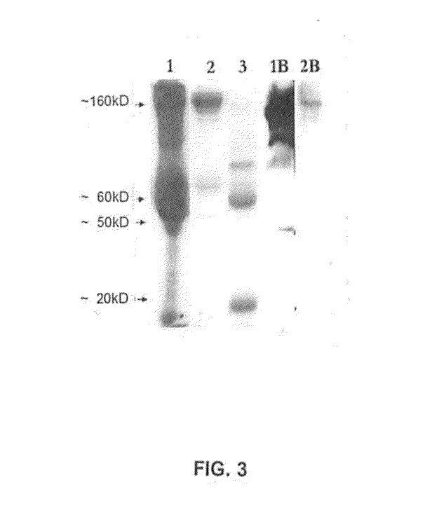

FIG. 3, lanes 1-3, is a photograph of a Coomasie blue stained SDS-PA gel run with G-protein affinity purified mAb3C4 from ascites. Lane 1 indicates significant quantities of mAb3C4 were released into the ascites as seen by the bulge around ˜150-160 kD region. Lane 2: low pH elution where IgG was quantitatively released from the bead, Lane 3 shows the ˜160 kD protein (IgG) of lane 2 reduced. The disappearance of the ˜160 kD protein and the appearance of ˜55 kD heavy and ˜28 kD light chains typically of IgG are evidence that the extracted 160 kD protein is in fact IgG. Lanes 1B and 2B depict immunoblots and autoradiograms (chemiluminescentograms) of the IgG in lanes 1 and 2 using HRP-SaM IgG and ECL reaction kit, confirming the ˜160 kD protein to be IgG. This purification resulted in extraction of about ⅔ of the antibodies present in the ascites and succeeded in removal of >98% of contaminants. ELISA analysis for isotype specificity identified mAb3C4 to belong to the IgG1 subclass of mouse IgG with kappa light chain.

### Example 9

### Identification of the 3C4 Antigen (PaCa-Ag1)

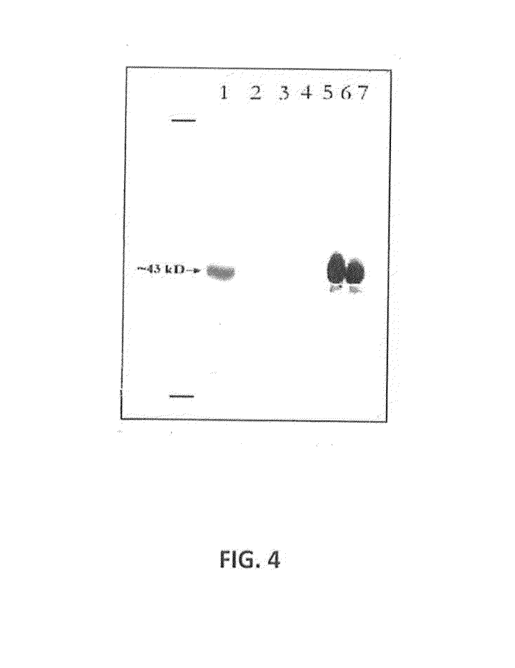

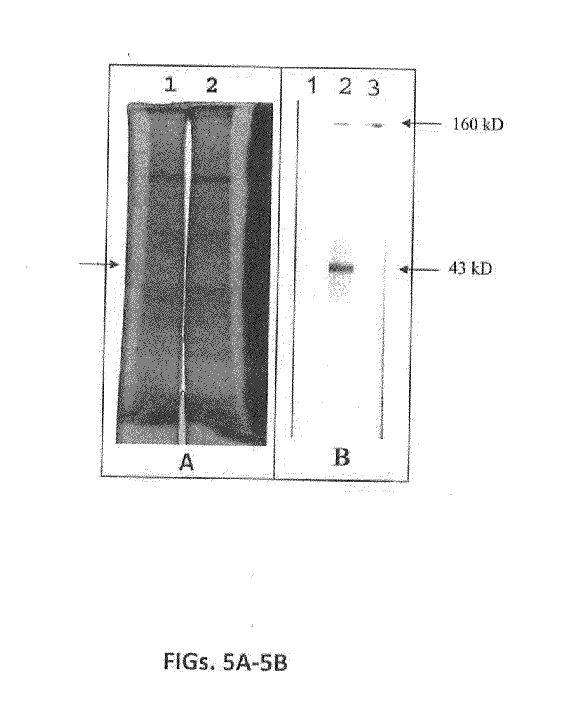

SDS PAGE of cell lysate proteins from rodent and human pancreatic carcinoma cells followed by IB with mAb3C4 was used to identify the protein nature and the molecular weight (MW) of 3C4-Ag (FIGS. 4 and 5). Cells were grown to confluence in 25 cm² TCDs, washed with ice-cold PBS, and incubated on ice with 0.5 mL RIPA lysing buffer (pH 8) consisting of 50 mM Tris-HCl, 1% NP40, 0.5% sodium deoxycholate, 0.1% SDS, 5 mM EDTA, 1 μg/mL pepstatin, 2 ug/ml, aprotinin, 1 mM PMSF, and 5 mM iodoacetamide. After 30 min, the remaining cell debris was scraped into the lysing solution, and the cell lysate was centrifuged at 11,500×g for 15 min to remove insoluble debris. The protein concentration of each lysate was determined by the Bradford's assay (BioRad). The cell extracts were mixed with equal volumes of non-reducing sample buffer (125 mM Tris-HCl, 2% SDS, 0.1% bromophenol blue, 20% v/v glycerol, pH 6.8) or reducing buffer (125 mM Tris-HCl, 2%(v/v) 2-mercaptoethanol, 2% SDS, 0.1% bromophenol blue, 20% v/v glycerol, pH 6.8). The proteins from each sample (20 μg/well) were separated by SIDS-PAGE as previously described (Laemmli, 1970), and electrotransferred onto nitrocellulose membrane. Gel lanes in FIG. 4 were loaded as follows:

### Lane Sample

1=BMRPA1.NNK+mAb3C4; with HRP-GαMIgG
2=BMRPA1+mAb3C4 with HRP-GαMIgG
3=BMRPA1.NNK without mAb3C4 but with HRP-GαMIgG:
4=BMRPA1.TUC3 with mAb3C4 with HRP-GαMIgG
5=non-reduced human MIA PaCa-2 without mAb3c4 but with HRP-GαMIgG
6=reduced MIA PaCa-2 without mAb3C4 but with HRP-GαMIgG;
7=reduced MIA PaCa-2 with mAb3C4 and with HRP-GαMIgG
8=non-reduced MIA-PaCa-2 with mAb3C4 and with HRP-GαMIgG
ECL amplification with HRP-GαMIgG.

### Horizontal Lines Indicated Top and Bottom of Separation Gels.

After the membrane was incubated with 5% (w/v) dry milk in TBS-T for 1 h. mAb3C4 (1:200) and the HRP-GαM IgG antibody were used as suggested by the manufacturer (ECL kit, Amersham). The presence of the protein of interest by ECL in each of the samples tested was detected by exposure to X-OMAT film (Kodak).

As shown in the immunoblot depicted in FIG. 4, the mAb3C4 clearly identified the 3C4-Ag to be about a 43-43.5 kD protein in the cell lysates of both rodent and human pancreatic carcinoma cells under both non-reducing (lanes 1-5, 8) and reducing (lanes 6 and 7) conditions. The protein is not present in lysates of normal, untransformed BMRPA1 cells present in NNK transformed cells and Human PaCa cell line MIA PaCa-2. The fact that reduction does not change the migration pattern of 3C4-Ag indicates that the antigen does not contain subunits.

FIG. 5 shows an immunoprecipitation of the 3C4 antigen firm BMRPA1.NNK cells with mAb3C4 and protein G immunoaffinity beads. In A, silver staining of protein gel shows the removal of a polypeptide band of about 43 kDa that is present in lane1 (protein G treated only) but absent in Lane 2 (treated with mAb3C4 and protein G beads. The extracted bands were identified in Lane 2 of FIG. 5B by immunoblotting with mAb3C4 as a single band of approximately 43 kDa.

### Example 10

### 2D Isoelectric Focusing/SDS-Duracryl Gel Electrophoretic Polypeptide Separation

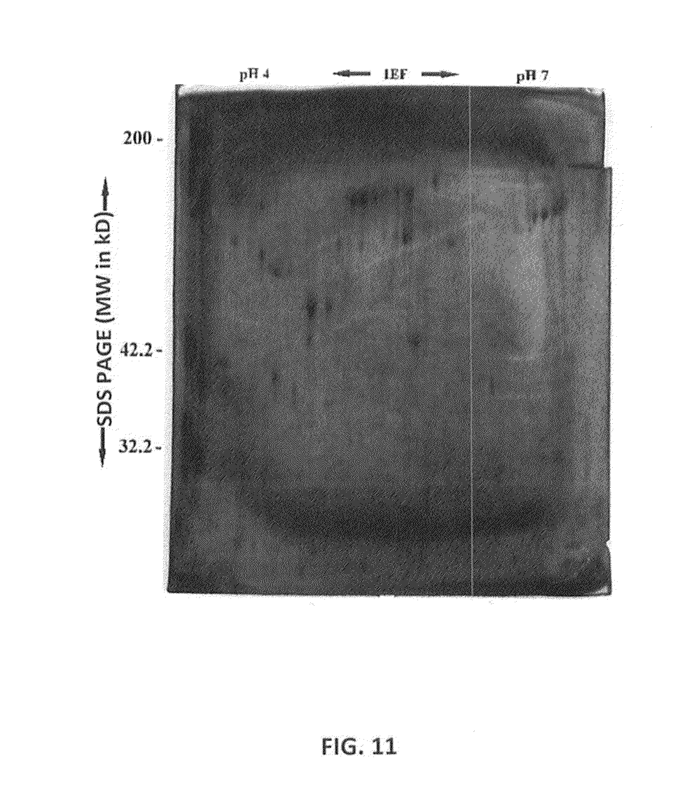

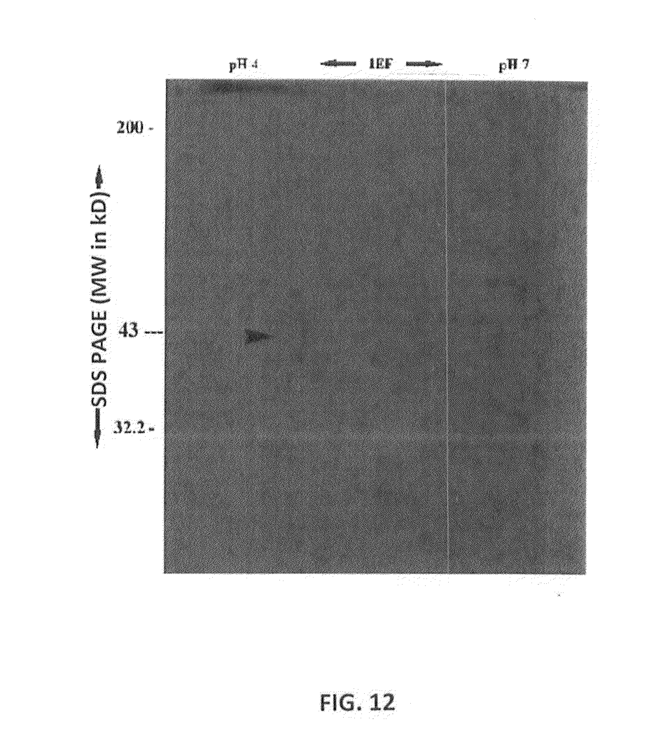

BMRPA1.NNK cells were lysed in situ in the presence of protease inhibitors, their nuclei removed by centrifugation, and the protein concentration of the cell lysate established by Bradford's assay (BioRad). Cell protein (0.4 mg) was transferred into isoelectric focusing sample buffer made with urea-/NP-40-solution (8.15 ml) and 2-mercaptoethanol (0.2 ml) in dH2O (1.65 ml) [urea-/NP-40 stock solution: 24 g urea dissolved in 18 ml dH2O containing 0.84 ml NP-40 (Nonidet)]. The lysate in sample buffer was then placed on top of IEF capillary tube gel consisting of acrylamide/bis-acrylamide (0.5 ml), urea-/NP-40 solution (3.76 ml), biolyte mixture (0.25 ml) ammonium sulfate (0.015 ml of 10% w/v solution) and TEMED (0.004 ml). Acrylamide/bis-acrylamide mixture was prepared with 9 g acrylamide and 0.54 g his-acrylamide dissolved in 30 ml dH2O. Biolyte (ampholine) mixture was made by combining Biolytes covering ranges from 3-10 (0.4 mil) and 5-7 (0.1 ml). Proteins were separated on the IEF gel for 2 h at 200V followed by 5 h at 500V and 16 h at 800V. The second dimension defining the molecular weights of the separated proteins was run in a 12% SDS-PAGE gel (BioRad) at 20 mA/gel. Several IF and SDS-PAGE gels were run in parallel under identical conditions and processed for silver staining (Genomic Solutions Inc.) (FIG. 11) and electrophoretic transfer to PVDF membrane (Schleicher and Scholl) for immunoblotting with mAb3C4 (FIG. 12) and to Immobilon membrane for the isolation of the 3C4-Ag spot for protein sequencing. Prestained molecular markers were used to verify appropriate transfer of the proteins from the IF gel to the membranes. The silver staining in FIG. 11 shows the presence of a large number of individual proteins in the cell lysate and their appropriate separation according to their PI values, within the IF gel. The immunoblot pictured in FIG. 12 was developed using the ECL-chemiluminescence procedure on X-ray film. The chemiluminescentogram of the mAb3C4 blot shows only a single spot of luminescence (arrow head) which identifies the 3C4-Ag as a ˜43 kD polypeptide with a pI of 4.6-4.8.

The separated polypeptides were either rapidly transferred onto a PVDF (Schleicher and Scholl) membrane under semi-dry conditions for one hour at 1.25 mA/cm² (484 mA), or, stained with a silver kit according to the manufacturer's instructions (Genomics Solutions, MA). The PVDF membrane was used for 3D4-Ag detection by Western blot analysis, and was later stained with either Rev Pro (Genomic Solutions, MA), or Amido Black. The pH gradient in the first dimension was determined from 1.0 cm sections as previously described (O'Farrell, 1975). The silver staining of the 2D separated polypeptides was recorded by computer scanning of the gel.

### Example 11

### Expression of the 3C4 Ag is Highly Restricted to Pancreatic Cancer Cells and Absent from Normal Tissues

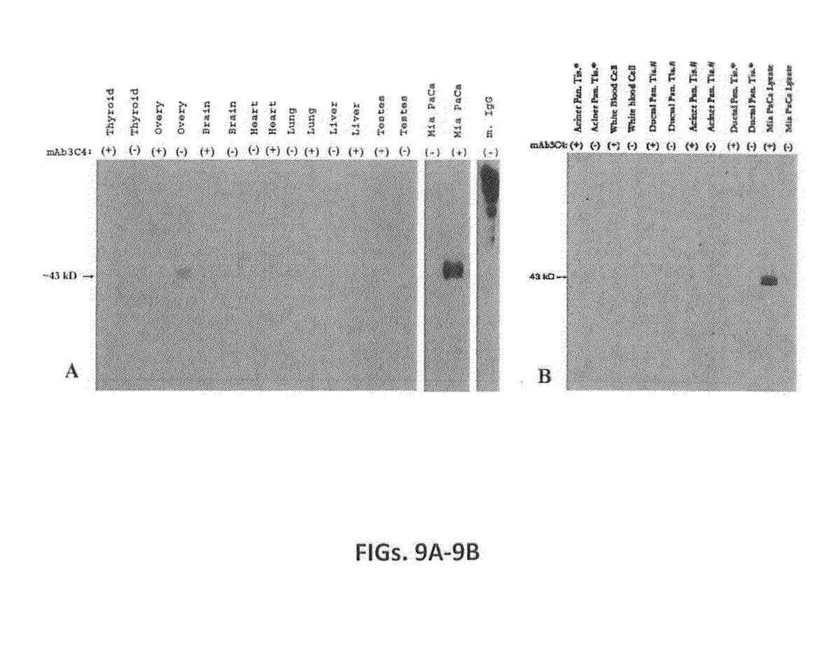

To examine the distribution of the 3C4-Ag within normal rat, human tissues and transformed human tissues, an immunoblot of tissue extracts using mAb3C4 was performed. Reduced proteins from tissue extracts from various tissues (thyroid, ovary, brain, heart, lung, liver, testes, see FIG. 9A) as well as human acinar pancreatic cells, white blood cells, and ductal pancreatic cells (see FIG. 9B) were separated on 12% SDS PAGE, electrophoretically transferred to nitrocellulose and processed with and without mAb3C4 followed by ECL chemiluminescence amplification. MIA-PaCa and mouse IgG served as controls. The extracts (0.05 mg/lane) of reduced proteins were separated on 12% SDS PAGE, electrophoretically transferred to nitrocellulose and processed with and without mAb3C4 followed by ECL chemiluminescence amplification (Amersham Pharmacia). Ten times and four times more protein of human pancreatic acinar (PA) and ductal tissues (PD) respectively, were loaded in order to rule out the presence of even minute quantities of the expression of the Ag. MIA PaCa-2 cell lysate and IgG were used as controls. Results as set forth in FIG. 9, indicate that the 3C4 Ag is absent from normal tissues but present in pancreatic cancer cells.

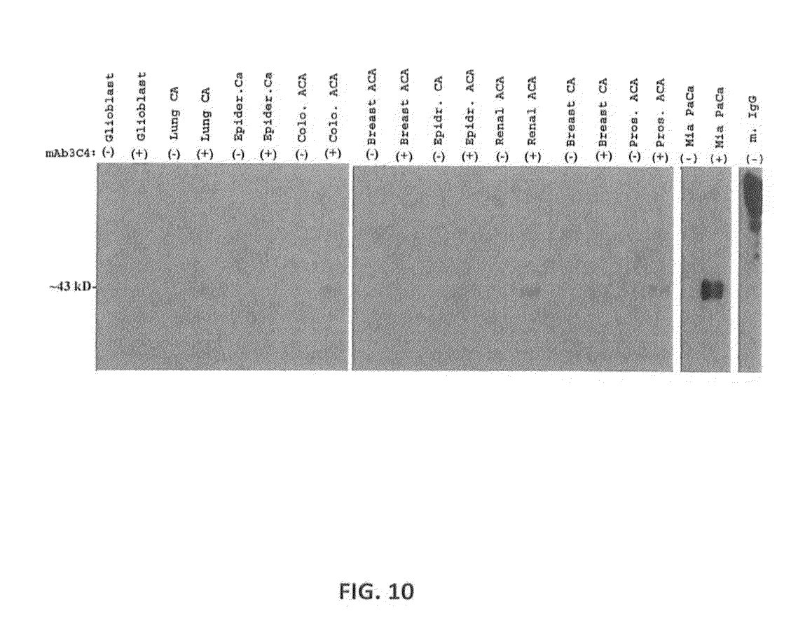

An immunoblot of various human cancerous tissue (glioblastoma, lung cancer, epidermal cancer, colorectal ACA, breast cancer ACA, epidermal ACA, renal ACA, MIA PaCa) using mAb3C4 was then performed, with the results set forth in FIG. 10. The results demonstrate a highly selective reactivity of mAb3C4 for an antigen of about 43.5 kD, the 3C4-Ag strongly expressed in human PaCa, MIA PaCa-2 cells. The specificity of the reactivity is further demonstrated by an absence of any protein hand in all tissue samples when mAb3C4 was omitted during the IB or replaced by non-specific IgG. There appears to be present small quantities of the 3C4-Ag in renal, prostate and possibly colon carcinoma, although the amount appears insignificant compared to the amount expressed by PaCa cells of which only 0.02 mg of protein were separated in the lanes shown. Taken together, the results obtained by IB and IC strongly support the specificity of mAb3C4 for an antigen, 3C4-Ag, that is preferentially expressed in rat and human PaCa cells.

Normal human pancreatic tissue (n=2) as well as purified human acinar and duct cells were found by western blot to be unreactive with mAb3C4. Furthermore, by Western blotting with mAb3C4, optimally preserved human tissue extracts (from Becton Dickenson) from tongue, esophagus, stomach, duodenum, ileum, jejunum, caecum, colon, brain, heart, trachea, lung, liver, kidney, mammary gland and prostate tissue and peripheral white blood cells were non-reactive tomAb3C4. Similar to rat ovary however, by Western blot with mAb3C4, a faintly positive 43.5 kDa band was observed with normal human ovary tissue.

### Example 12

### Further Studies on Characterization, Tissue Distribution, and Relative Expression Levels of PaCa-Ag1

Immunocytochemistry and Indirect Immunofluorescence (IIF) of transformed cells but not of untransformed cells fixed in either paraformaldehyde or methanol/acetone displayed accentuated staining of membranes. Cells were cooled on ice prior to reaction with mAb3C4 followed by FITC-GaMIgG and fixation in buffered 2% paraformaldehyde. A, B, C and D×40 objective: E, F×64 objective; Fuji 400 ASA film.

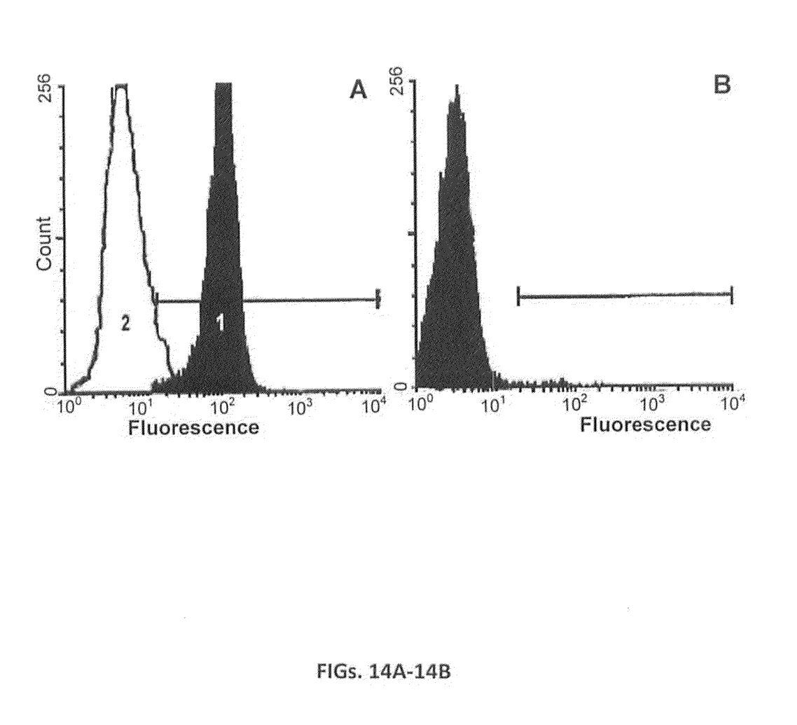

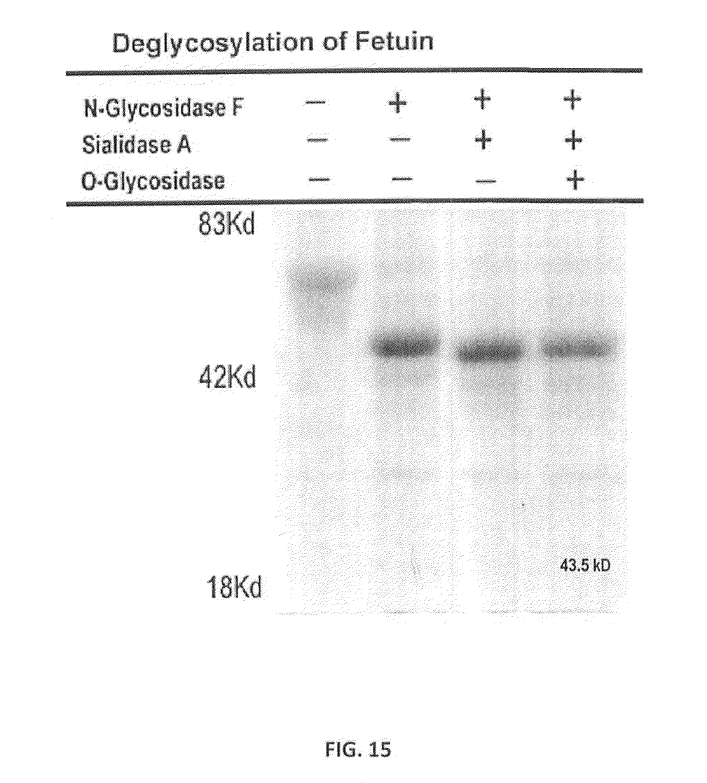

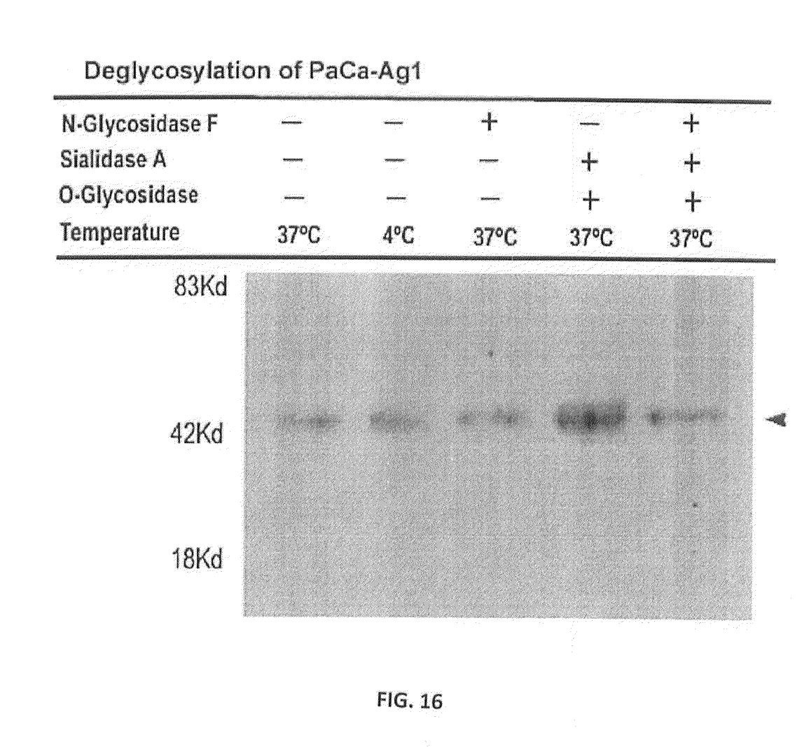

Trypsin digestion of whole cells resulted in degradation of the PaCa-Ag1 protein, consistent with a location on the plasma membrane (FIGS. 14A-14B). However, exposure to exo- and endoglycosidases (Prozyme) (Iwase et al., 1993; Altmann et al., 1995; Lee and Pack, 2002) neither eliminated antigenicity nor changed to any appreciable extent the electrophoretic mobility (FIG. 15), indicating that PaCa-Ag1 is not or is only minimally glycosylated, and that the epitope on PaCa-Ag1 recognized by monoclonal mAb3C4 is likely to be a pure peptide rather than a carbohydrate-containing region. This may reduce the likelihood of cross-reactivity that carbohydrate-containing epitopes may be more subject to, compared to peptide epitopes.

PaCa-Ag1 was found to be an abundant protein: Using fluorescein isothiocyanate (FITC)-labeled mAb3C4 and cytofluorimetry (FACS) in the presence of beads carrying standardized amounts of the fluorophore (QuickCal Quantum-26, Bangs Lab) (Zagursky et al. 1995, Borowitz et al, 1997, Schwartz et al, 1998), it was determined that transformed BMRPA1 cells expressed 2-4.4×10⁵ copies of PaCa-Ag1 per cell. Reactivity to mAb3C4 was nil in untransformed BMRPA1 cells by immunofluorescence and immunoblot and nil in normal rat pancreas by immunoblot (FIGS. 9 and 10). Moreover, no mAb3C4-reactive protein was detectable in normal rat oral squamous epithelium, esophagus, stomach, small intestine, large intestine, liver (comprising hepatocytes and bile duct epithelium), lung, heart, thyroid, testes, brain and peripheral blood cells.

The only normal rat tissue with mAb3C4 reactivity was mature ovary, which displayed trace reactivity of an approximately 43.5 kD protein.

TABLE 3
Human cell lines and tissues tested for expression of PaCa-Agl
Neoplastic Cell Lines

| | | Reactivity | |
|---|---|---|---|
| Name | Origin | Western Blot | Fluorescence |
| MIA PaCa-2 | Pancreatic Cancer | +++ | +++ |
| BxPC-3 | Pancreatic Cancer | +++ | +++ |
| Capan-1 | Pancreatic Cancer | +++ | +++ |
| | (metastatic) | | |
| Capan-2 | Pancreatic Cancer | +++ | n.d. |
| | (metastatic) | | |
| A431 | Epidermoid Cancer | 0 | n.d. |
| A549 | Non-small cell lung cancer | ± | 0 |
| BT-20 | Breast Carcinoma | 0 | n.d. |
| MDA-MB-231 | Breast Carcinoma | 0 | n.d. |
| U-87 | Glioblastoma | 0 | n.d. |
| COLO320 DM | Colorectal Carcinoma | 0 | n.d. |
| LNCaP | Prostate Carcinoma | ± | n.d. |
| HeLa | Cervical Cancer | 0 | n.d. |
| | Normal Tissues | | |
| | Pancreas (2x) | 0 | n.d. |
| | Pancreatic Aciner Cells (2x) | 0 | n.d. |
| | Pancreatic Ductal Cells (2x) | 0 | n.d. |
| | Peripheral | 0 | 0 |
| | Brain | 0 | n.d. |
| | Tongue | 0 | n.d. |
| | Esophagus | 0 | n.d. |
| | Stomach | 0 | n.d. |
| | Duodenom | 0 | n.d. |
| | Ileum | 0 | n.d. |
| | Jejunum | 0 | n.d. |
| | Caeccum | 0 | n.d. |
| | Colon | 0 | n.d. |

### Example 13

### Demonstration of Complement-Mediated Cytotoxicity of mAb3C4 to PaCa Cells

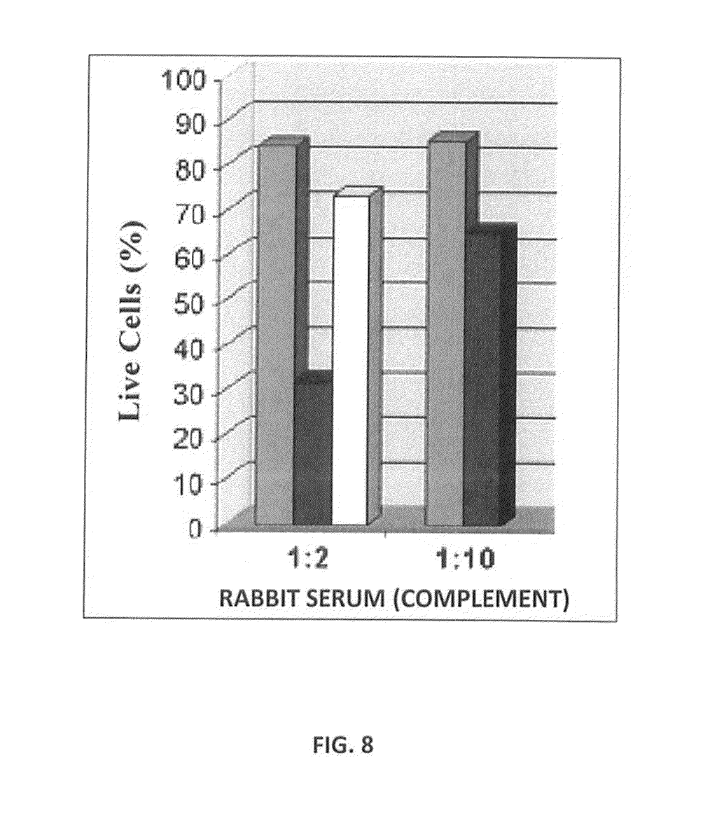

The Cylotoxicity of mAb3C4 was determined as follows: Human MIA PaCa-2 cells were incubated with mAb3C4 at 4° C. followed by incubation in fresh rabbit serum as a source of complement (C) at 37° C. The results, set forth in FIG. 8, show that with increasing concentration of C at a constant concentration of mAb3C4, an increasing number of cell lysis was obtained. In contrast, even at the highest concentration, HI-C (HI-C=Heat inactivated rabbit serum, 56° C., 45 mins) was equally ineffective in demonstrating cytotoxicity towards MIA PaCa-2 cells as was C in the absence of mAb3C4. Similar results were obtained for BMRPA1.NNK and BMRPA1.Tuc3 cells used in this assay. All dilutions and reactions were made in PBS containing Ca⁺⁺ and Mg⁺⁺.

### Example 14

### Effect of mAb3C4 on Tumor Growth In Vivo

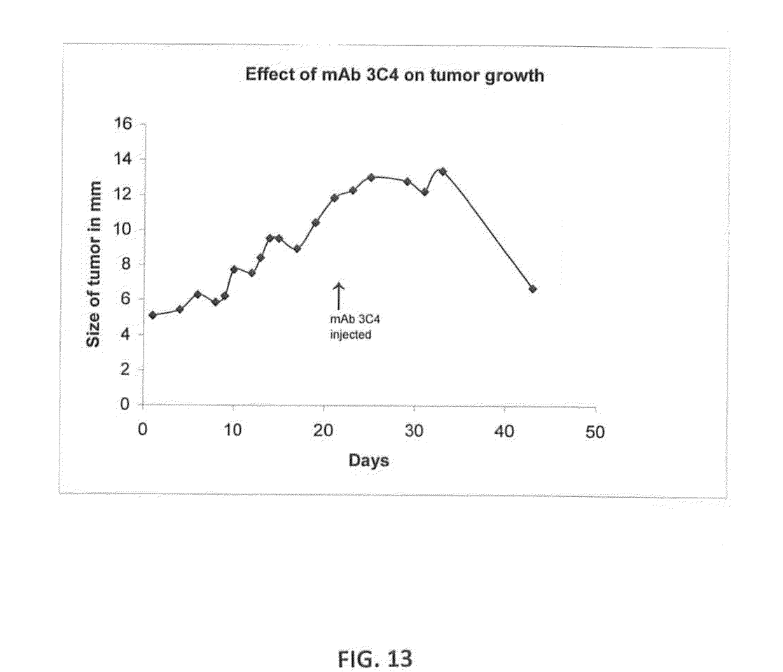

Nu/Nu mice (n=10) were xenotransplanted with BMRPA1.TUC3 cells (5×10⁶ cells/mouse) subcutaneously. Tumors were allowed to develop and grow until they reached diameters of from 10 to 14 mm. At this time, 3C4 hybridoma cells secreting mAb3C4 were injected intraperitoneally (ip) at 10⁶ cells per mouse. Subsequently, at 2 day intervals, tumor development was observed and the diameter of tumors measured. Within 4 days, tumor growth was arrested and within 16 days, tumor size regressed to values of between 4-6 mm in diameter, i.e., significantly below the size measured initially at the time of 3C4 hybridoma IP injection. See FIG. 13. Significance value of tumor regression is <0.00066 as determined using mixed model analysis.

### Example 15

### Construction of Adenoviral Vectors with High Specificity for 3C4-Ag Presenting Cells

### Ad Vector Construction:

Two single stranded DNA fragments were synthesised by Invitrogen with a DNA sequence corresponding to the peptide sequence published by (Kanovsky et al., 2001). In addition to the peptides sequence it also contained a start codon, a Kozak motif, a stop codon and two restriction sites for Not1 and Kpn1 and additional 4 base pairs on each end to allow the restriction enzyme to bind properly.

Sequences were:

(SEQ ID NO: 5)

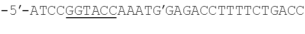

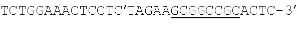

5′ enzyme: Kpn1; 3′ enzyme: Not1
(SEQ ID NO: 6)
-3′-TAGGCCATGGTTTAC′CTCTGGAAAAGACTG
GAGACCTTTGAGGAG′ATCTTCGCCGGCGTGAG-5′

3 μg GOI-frw and 3 μg of GOI-rev were mixed with 2.5 μl 10×PCR Buffer (Qiagen), 0.5 μl dNTPs (10 mM each, Qiagen), 0.5 μl of Taq polymerase (Qiagen) and 19.5 μl of sterile water to a total reaction volume of 25 μl. The sample was denatured at 94° C. for 5 minutes (′), let anneal at 50° C. for 1′ and incubated at 72° C. for 10′.

### Cloning and Transfection of Bacteria:

4 ul of the above reaction were taken for TA-cloning reaction were added to chemically competent TOP-10 one shot E. coli (Invitrogen). Bacteria permiabilated at 42° C. for 30 seconds (″) and incubated in SOC (Invitrogen) medium (Invitrogen) for 1 h at 37 C. Bacteria were plated on selective LB-agar plates containing Kanamycin (50 μg/ml) and incubated at 37° C. over night.

### Analysis of Bacterial Clones:

12 colonies were selected at random and grown in liquid culture (LB medium containing 50 μg/ml Kanamycin) over night. Bacteria from 3 ml culture medium were then harvested and a plasmidisolation was performed using Qiagen's Miniprep plasmid isolation Kit, 10 μl of each isolated plasmid were digested with 10 units (U) EcoR1 restriction enzyme for 1 h at 37° C. and half of each of the digested plasmid analyzed on a 2% agarose gel. Plasmids showing an insert of the expected size were sent for sequencing to Genewiz Inc., NJ.

### Construction of Entry Vector:

40 μg of plasmid containing the expected sequence were digested in a 50 ul reaction volume with 40 U Not1 (NEB) at 37 C for 1 h. Then half the volume Phenol:Chloroform=1:1 was added, sample vortexed and centrifuged at maximum speed for 3′. The top layer was transferred into a new tube and precipitated with 3M sodium acetate solution and 100% Ethanol (Sambrook et al., 1989). The Plasmid was re-eluted and digested with 40 U Kpn1 (NEB) in a 50 μl reaction volume at 37° C. for 1 h, 40 μg of the vector pENTR11 (Invitrogen) were processed in parallel. Both reactions were analyzed on a 2% agarose gel and then the entire mixture was run on a 1% agarose gel. Appropriate bands were excised and extracted from the gel using the Gel Extraction Kit from Qiagen. Since the maximum binding capacity of one column contained in the kit is 10 ug of DNA, the digested pENTR11 reaction was split up in three fractions and processed separately, then pooled again. OD of the samples were taken and a ligation reaction using T4-DNA ligase (NEB) with the appropriate concentration of 5′ termini was incubated for 4 h at 16 C (Sambrook et al., 1989), 4 ul of the ligation reaction were used to transfect E. coli as described above, 12 colonies were analyzed for presence of GOI and a positive clone chosen for the successive experiment.

Construction of Adenoviral vector containing PNC-28 (Ad/CMV/V5/PNC-28):

300 ng of pENTR11-PNC-28 and the same amount of Ad/CMV/V5 vector were used in a lambda recombination reaction as described in the manufacturers protocol and incubated for 2 h at 25 C (Invitrogen, Carlsbad, Calif.).

### Propagation of (Ad/CMV/V5/PNC-28) in 293A Cells:

1 ul of the above reaction mixture now containing Ad/CMV/V5/PNC-28 was transfected into TOP-10 chemically competent E. coli and grown on Ampicillin plates (100 ug/ml). Colonies were selected and it was attempted to grow them in LB-chloramphenicol (30 ug/ml). If this failed, as it should in a true positive clone, the bacteria were propagated in LB-ampicillin (100 ug/ml) and isolated as described above. To transfect 10⁶ 293A cells were plated in 2 ml normal growth medium in a six well dish per well per transfection, 4 ug of the vector were digested with 4 U Pac1 (NEB) in a 50 ul reaction volume at 37 C for 1 h, phenol:chlorofrom extracted, precipitated as described above and eluted at a concentration of 1 ug/ul, 18 h post plating the cell's medium was substituted with antibiotic free normal growth medium, 24 h post plating cells were transfected a Ad/CMV/V5/PNC-28—Lipofectamine 2000) (Invitrogen) at a DNA: Lipofectamine 2000 ratio of 2:5 in 0.5 ml of antibiotic and FBS free OPTI-MEM medium (Invitrogen), 24 h post transfection the medium was replaced by normal growth medium containing antibiotics and FBS, 48 h post transfection cells were transferred into 10 cm² dishes, fed every 2 days until 60% cytopathic effect (CPE) was observed and viruses were harvested according to manufacturers protocol once 80% CPE was reached.

cDNA Synthesis from 293A and 3C4-Hybridoma Cells:

3×10⁷ cells were collected of each cell line. Total RNA was isolated using the Rneasy minikit (Qiagen) and poly-A⁺ mRNA isolated using Clontech's Nucleotrap mRNA purification kit, 1 ug purified mRNA of each cell line was used to synthesize DNA using the SMART PCR cDNA Synthesis kit (Clontech). The cDNA was analysed on a 1% agarose gel to verity its integrity.

### PCR-Amplification of the Exoplasmatic Region of CAR:

The sequence of the human CAR was viewed on www.ncbi.nih.gov and primers flanking the exoplasmatic region plus a Sfi1 (5′) and a Not1 (3′) restriction site were synthesized (Invitrogen). Sequences are as follows:

(SEQ ID NO: 7)
-5′-atcc′ggcccagccggcc′gcgctcctgctgtgcttcgtg-3′
Sfi1 CAR-frw
(SEQ ID NO: 8)
-5′-atcc′gcggccgc′agcgcgatttgaaggagggac-3′
Not1 CAR-rev

A PCR was carried out as follows, 10 pmol of each primer were mixed with 2.5 ul 10×PCR Buffer (Qiagen), 0.5 ul dNTPs (10 mM each, Qiagen), 0.5 ul of Taq polymerase (Qiagen) and 19.5 ul of sterile water to a total reaction volume of 25 ul (Saiki et al., 1985). The cycling conditions were 95 C, 5′, (95 C, 1′; 60 C, 1′; 72 C, 2′)×30, 72 C, 10′. The PCR product was subjected to TA cloning (TA cloning kit, Invitrogen), clones analysed and sequenced as described above.

PCR-Amplification of the Variable Regions of Heavy (V_{H}-3C4) and Light Chain (V_{L}-3C4) of mAb-3C4:

Primer consisting of the constant flanking regions of the variable regions of heavy and light chain were purchased from Novagen. PCR's to amplify V_{H}-3C4 and V_{L}-3C4 were carried out as suggested by the company using Advantaq polymerase mix (Clontech).

The PCR product was subjected to TA cloning (TA cloning kit, Invitrogen), clones analysed and sequenced as described above. New primers were designed to match the obtained sequences that contained additional restriction sites to allow proper insertion into an expression vector. Primes were synthesized by Invitrogen (Carlsbad, Calif.). Primer sequences were:

V_{H}:
(SEQ ID NO: 9)
frw: -5′-atcc′gcggccgc′-3′
Not1
(SEQ ID NO: 10)
rev: -5′-atcc′cctagg′-3′
BamH1
V_{L}:
(SEQ ID NO: 11)
frw: -5′-atcc′ggatcc′t′ggt′atggagacagacacactc-3′
BamH1
(SEQ ID NO: 12)
rev: -5′-atcc′ctcgag′c′tttccagcttggtccccc-3′
Xho1

A PCR was carried out as follows, 10 pmol of each primer were mixed with 2.5 μl 10×PCR Buffer (Clontech), 0.5 ul dNTPs (10 mM each, Clontech), 0.5 μl of Taq polymerase (Clontech) and 19.5 μl of sterile water to a total reaction volume of 25 μl. The cycling conditions were 95° C., 5′, (95 C, 1′; 55 C, 1′; 72 C, 2′)×30, 72 C, 10′. The PCR product was subjected to TA cloning (TA cloning kit, Invitrogen), clones analyzed and sequenced as described above. Clones containing the desired sequence were selected for the construction of an expression vector.

Construction of a Eukaryotic Expression Vector Containing CAR, V_{H}-3C4 and V_{L}-3C4:

40 μg of plasmid containing the expected sequence for CAR were digested in a 50 μl reaction volume with 40 U Not1 (NEB) at 37° C. for 1 h. Then half the volume Phenol:Chloroform=1:1 was added, sample vortexed and centrifuged at maximum speed for 3′. The top layer was transferred into a new tube and precipitated with 3M sodium acetate solution and 100% Ethanol. The Plasmid was re-eluted and digested with 40 U Sfi1 (NEB) in a 50 ul reaction volume at 50 C for 1 h, 40 μg of the chosen eukaryotic expression vector pSecTag2A (Invitrogen) were processed in parallel. Both reactions were analysed on a 2% agarose gel and then the entire mixture was run on a 1% agarose gel. Appropriate bands were excised and extracted from the gel using the Gel Extraction Kit from Qiagen. Since the maximum binding capacity of one column contained in the kit is 10 ug of DNA, the digested pENTR11 reaction was split up in three fractions and processed separately, then pooled again. OD of the samples was taken and a ligation reaction using T4-DNA ligase (NEB) with the appropriate concentration of 5′ termini was incubated for 4 h at 16° C., 4 ul of the ligation reaction were used to transfect E. coli as described above, only that the antibiotic was Ampicillin (100 ug/ml), 20 colonies were analyzed for presence of CAR via PCR screening. For this experiment 1 reaction tube per colony was prepared as described for the PCR amplification of CAR above except it did not contain template. Cycling conditions were as mentioned previously. The PCR products were analysed on a 2% agarose gel and a positive from now on designated pSecTag2A-CAR², clone chosen for the successive experiment.

This procedure was repeated for V_{L}-3C4 using the restriction enzymes as indicated above and the plasmid designated pSecTag2A-V_{L}-3C4.

To prepare the final construct 40 ug of TOPO-TA vector containing V_{H}-3C4 was digested with BamH1 and Not1, while 40 ug of each vector (pSecTag2A-CAR² and pSecTag2A-V_{L}-3C4) were digested with Not1 and BamH1 respectively as described previously and an intermediate construct obtained by ligating V_{H}-3C4 between the two other genes, both flanked on one side by now linearized pSecTag2A vector. This construct was digested with Xho1, gel purified and ligated into an expression vector now designated pSecTag2A-CAR²-V_{K}-3C4-V_{L}-3C4 as described above.

### Example 16

### Detection of a Soluble Form of PaCa-Ag in Rodent and Human Samples

Ascites collected from intraperitoneal (i.p.) implants of ras-transformed subline BMRA1.TUC3 cells (n=3) in athymic mice as well as ascites formed in athymic mice implanted s.c. with these cells (n=2) displayed a soluble form of PaCa-Ag1: a mAb3C4-reactive protein of molecular weight 36-38 kD. In contrast, control ascites induced by i.p. implantation of P3U-1 mouse myeloma cells contained no mAb3C4-reactive protein.

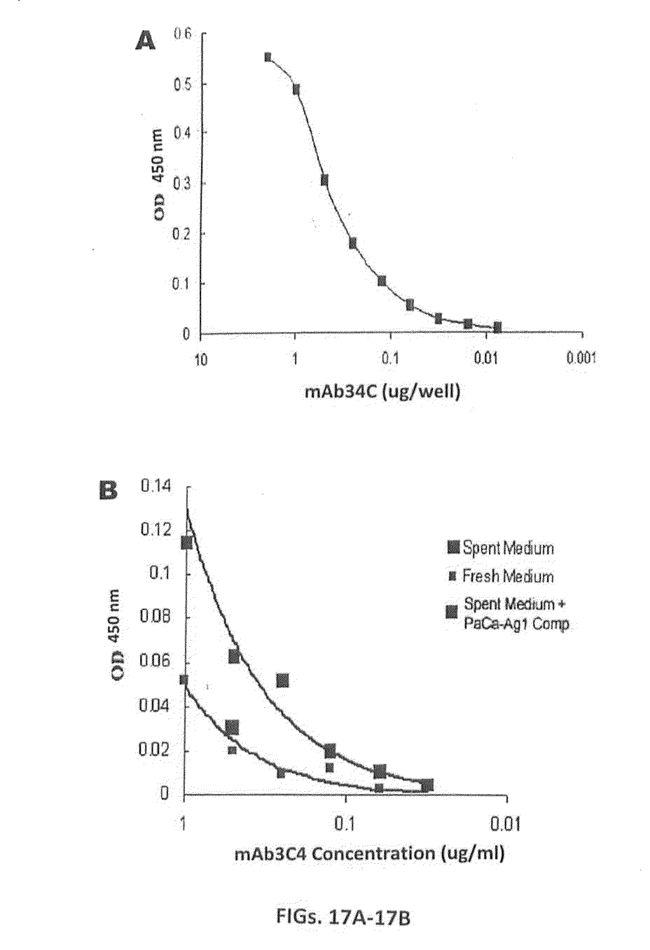

Similarly, sera and ascites from mice that had been xenotransplanted s.c. with BMRPA1.TUC3 and that had grown tumors of 256-1220 mg were found positive by one-antibody antigen-adsorbance ELISA for binding of mAb3C4 to the wells of 96-well plates to which the serum proteins had adsorbed (FIG. 17C). The one-antibody antigen-adsorbance ELISA uses mAb3C4 to locate and bind to the PaCa-Ag1 present in a well, and a second. HRP (horse radish peroxidase)-labeled sheep-anti-mouse IgG (HRP-S□MIgG) followed by the HRP substrate TMB (tetramethylbenzidine) and measuring absorbance at OD₄₅₀ₙₘ.

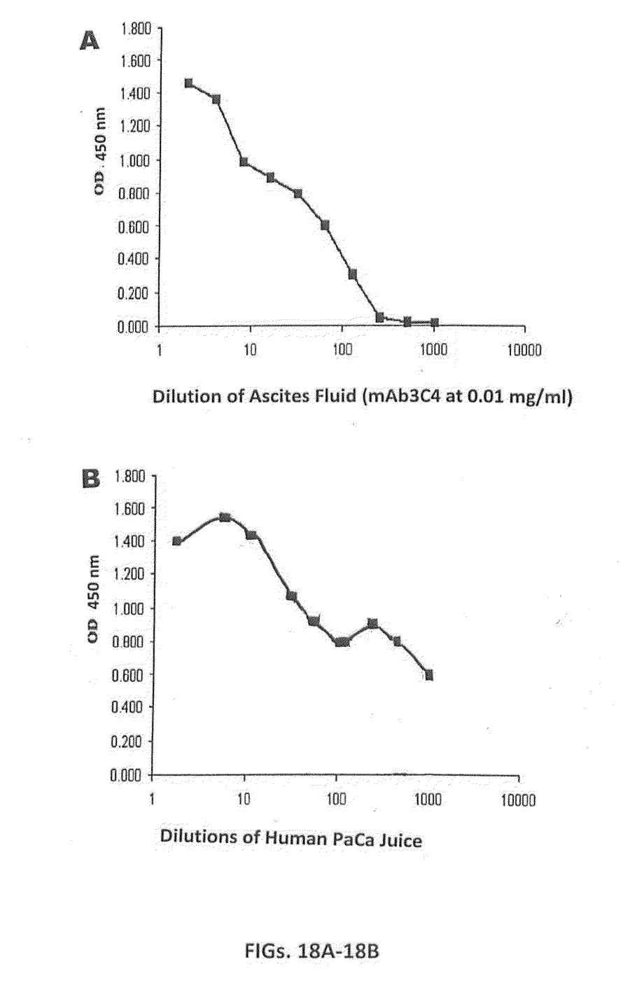

FIG. 17A shows the titration of mAb3C4 concentration of semi-pure PaCa-Ag1. Inserts show electroeluted PaCa-Ag (n=2). In FIG. 17 B. PaCaAg1 is present in spent (18 h) cell culture media (not conc.) of pancreatic cancer cells (BMRPA.NNK). The red square shows effective competition at half maximal binding of mAb3C4 binding to adsorbed PaCa-Ag1 by soluble PaCa-Ag1 (n=²). FIG. 18A shows the presence of PaCa-Ag1 in ascites of mice xenotransplanted with pancreatic carcinoma BMRPA.TUC3 cells (n=5) but not in control ascites (not shown) after P3U1 transplantation (n=2). FIG. 18B shows PaCa-Ag1 in pancreatic duct juice (ERCP) of pancreatic cancer patient (n=1). Background measurements of control wells were subtracted.

The presence of measurable amounts of PaCa-Ag1 in tissue culture fluids of transformed BMRPA1 and human MiaPaca-2 cells was demonstrated by one-antibody antigen-adsorbance ELISA (FIG. 17B). Cell viability was >98%, minimizing the likelihood that ELISA positivity was caused by disintegration of cells rather than release of the PaCa-Ag1, or fragment thereof, by living cells.

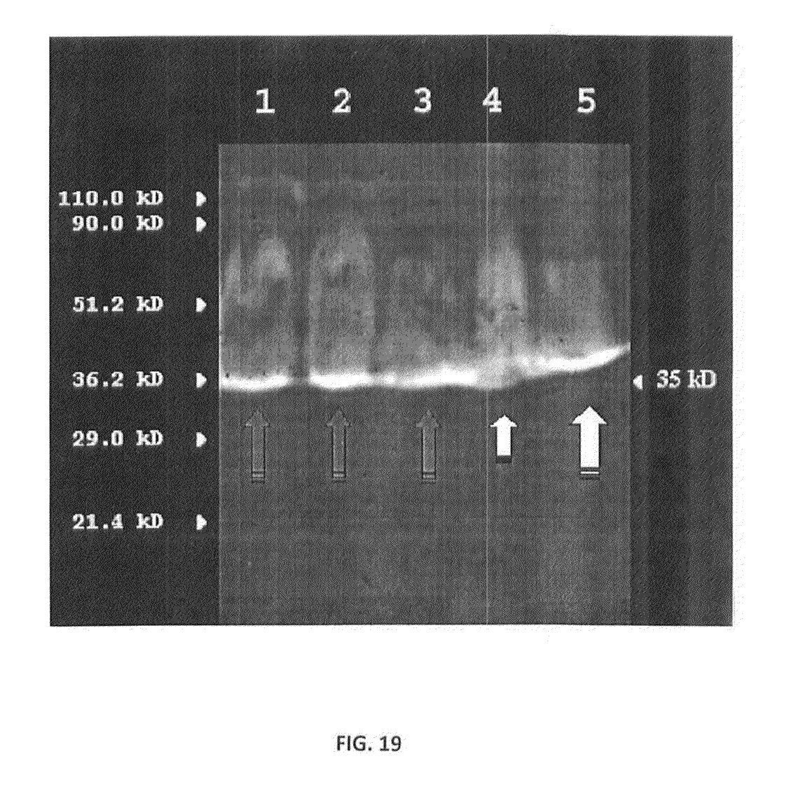

Serum samples from three patients with pancreatic adenocarcinoma were examined by Western blot for reactivity to mAb3C4. All three sera displayed robust reactivity to mAb3C4, consisting of a single protein of molecular weight (MW) 36-38 kD (FIG. 19. Lanes 2-4) that is essentially the same MW as the soluble form of PaCa-Ag1 found in mouse ascites. A serum sample from a healthy human control showed no reactivity with mAb3C4. A pancreatic duct secretion sample obtained during endoscopic retrograde cholangiopancreatography (ERCP) in a patient with known pancreatic adenocarcinoma also revealed the presence of a protein reactive with mAb3C4. This was demonstrated with a one-antibody antigen-adherence ELISA: PaCa-Ag1 was present in the wells to which the proteins in the ERCP fluid had been allowed to adsorb for a defined time (FIG. 18B).

### Example 17

### Separation and Purification of PaCa-Ag1

Consistent with other findings, cell fractionation of neoplastically transformed rat BMRPA1 cells and human MIAPaCa-2 pancreatic cancer cells has revealed PaCa-Ag1 to be found exclusively in the membrane soluble fraction, not in the particulate or nuclear fractions. PaCa-Ag-1 has also been identified with mAb3C4 in non-denaturing electrophoretic and iso-electric focusing gels. Electro-eluted 43.5 kD PaCa-Ag1 but not proteins of larger or smaller molecular size has been shown to compete effectively and dose-dependently with mAb3C4 binding to PaCa-Ag1 on pancreatic carcinoma cells and to antigen protein in the one-antibody (mAb3C4) antigen (PaCa-Ag1)-adsorbance ELISA. Based upon these findings, PaCa-Ag1 from plasma membrane fractions of human MiaPaCa-2 pancreatic carcinoma-derived cells may be immunoprecipitated, the PaCa-Ag1 protein separated electrophoretically from any contaminants and electroeluted for mass spectroscopic identification of its amino acid (AA) sequence.

Method:

The availability of the PaCa-Ag1-specific mAb3C4 makes feasible immunoaffinity extraction of PaCA-Ag1 from cell lysates as a direct approach to isolate the 43.5 kD polypeptide. Mia-PaCa-2 cells may be used for isolation of the PaCa-Ag1 protein, since these human pancreatic carcinoma-derived cells express 10× more PaCa-Ag1 on the plasma membrane than is expressed by rodent pancreatic carcinoma cells BMRPA1.NNK and BMRPA.TUC3. For the actual affinity approach to cell fractionation and membrane protein isolation the procedures described previously in Schneider et al., (1982); and Deissler et al., (1995, may be used.

In preparation for the immunoaffinity extraction of PaCa-Ag1 from the solubilized membrane fraction, 4-8 mg of affinity-purified mAb3C4 may be crosslinked in the presence of dimethyl pimelimidate (DMP, 0.1 M) in sodium borate buffer (0.1M, pH8.2) to 1 ml of Protein G beads (Amersham-Pharmacia) (Schneider et al., 1982). Samples of the mAb3C4-derivatized beads may be analyzed by SDS-PAGE for irreversibly bound antibody. The ready-to-use mAb3C4-Protein G beads may be resuspended to a 50% suspension in solubilization buffer (see below) for immediate use. Plain Protein G beads will be processed in parallel in the absence of any mAb.

From a mass culture of MIA PaCa-2 (about 10 cells, 30-40 large tissue culture flasks), cells at 80-90% density may be collected and washed, pelleted at 250×g, resuspended (10× the cell volume) in homogenization buffer [NaPO₄ (0.02 M) pH7.4, sucrose (0.25M), protease inhibitors cocktail 1:100 (Invitrogen)] and subjected to homogenization for 2 min in ice at 30,000 rpm in an Omni homogenizer (Omni). After centrifugation (1000×g) of the homogenate (precipitate 1=P1, and supernatant 1=S1) the S1 may be collected and subjected to ultracentrifugation at 140,000×g, 1 h, for the separation of the insoluble membrane fraction in the pellet (P2) (that contains PaCa-Ag1) from the fraction of soluble proteins (S2). The pellet is washed once by ultracentrifugation (30,000×g, 30 min) and resuspended directly in solubilization buffer (Tris-HCl (0.04M) pH7.5, NaCl (0.2M), CaCl2 (0.001M), MgCl2 (0.001M), n-octyl-b-d-glucoside (0.05M, deoxycholate (0.14%), protease inhibitors cocktail 1:100] for immunoaffinity extraction of the PaCa-Ag1. Protein samples (0.05 mg protein) from steps P1, S. S2 and P2 collected during cell homogenization can be examined by SDS-PAGE (Laemmli, 1970) for differential protein patterns indicative of effective cell fractionation (Beaufy et al, 1976).

Proteins may be released from the membranes by incubation in solubilization butter containing n-octyl-b-d-glucoside (0.05M) in Tris-HCl (0.04M, pH7.5), 0.2M NaCl, CaCl₂ (0.001M), MgCl₂ (0.001M), deoxycholate (0.14%), and protease inhibitors cocktail for 1.5 h with frequent vortexing. Preliminary tests to ascertain the use of a particular protein solubilization-buffer have shown that n-octyl-b-d-glucoside releases about 2× the amount of PaCa-Ag1 from the tumor cells than is released during the same time period by Triton X-100. After the 1.5 h release period, the soluble fraction which contains the solubilized proteins can separated from the insoluble material by ultracentrifugation at 100,000×g. The amount of protein recovered is measured by OD₂₈₀ₙₘ readings or using the colorimetric assay BioRad Protein assay. A small quantity may be set aside for SDS-PAGE and for verification of the protein content, and the presence of PaCa-Ag1 by Western blot. The actual extraction may be performed by adding 0.05 ml of mAb-3C4 to each 0.2 ml of protein extract, and continued incubation for up to 1 h. Control-beads may be processed with a similar amount of cell protein. After extensive washing of the beads with solubilization buffer, bound protein can be released by incubation with a low pH releasing-buffer (glycine 0.01 M, pH 2.8) which requires that each fraction collected be immediately neutralized by adding a precise amount of basic phosphate buffer (Na₃HPO₄ 0.1M, pH12). The protein content of each sample may be measured, and a fraction analyzed by SDS-PAGE followed by silver staining and/or Western blot. As an alternative to low pH release, the affinity-bound PaCa-Ag1 can also be released by basic triethanolamine at pH 12 (Deissler et al., 1995).

Once the PaCa-Ag1 is released, it may be concentrated by vacuum centrifugation and the concentrate examined by SDS-PAGE to confirm that its purity is sufficient to be processed for AA analysis by mass-spectroscopy. If the purity of the protein is still low, the PaCa-Ag1 can be further purified by 2-D gel separation in which another step of separation by isoelectric focusing is added (O'Farrell, 1975). The location of the PaCa-Ag1 protein spot in the gels may be identified by Western blot using mAb3C4 on one of six replicate gels.

### Example 18

### Development of Sandwich ELISA

In contrast to a one-Ab Ag-adsorption assay, the two antibody or “sandwich” ELISA enables one to make at once and under precisely defined conditions, a large number of 96-well ELISA plates to which a known amount of an Ab specific for PaCa-Ag1 is bound to the well surfaces. Since the amount of anti-PaCa-Ag1 Ab bound per well can be measured, the optimal amount of the anti-PaCa-Ag1 (the capture Ab) can be titrated with purified PaCa-Ag1 to establish reaction conditions for PaCa-Ag1 that will allow the measurement of pico molar amounts of PaCa-Ag1 protein in sera of patients with pancreatic carcinoma. To complete the measurements in the “sandwich” ELISA of PaCa-Ag1 the existing well-defined mAb3C4 can be used in combination with a second HRP-SαMIgG, if the Ab in the wells that captures the PaCa-Ag1 from the sera is not from mouse but another species (Ito et al., 2002; Plested et al., 2003).

Additional hybridomas that react with BMRPA1.NNK and BMRPA1.TUC3 cells but not with untransformed BMRPA1 cells may be analyzed for the presence of mAb reactive with purified PaCa-Ag1 by Western blotting (see above).

Those identified as reactive with PaCa-Ag1 may then be examined for possible binding to the same epitope to which mAb3C4 binds. Competition assays of the newly identified mAbs with mAb3C4-binding to PaCa-Ag1 in Western blots will enable the identification of those mAbs that bind directly or close enough to the mAb3C4 epitope to prevent binding of mAb3C4 to the PaCa-Ag1. These mAb will not be useful in the “sandwich” ELISA assay. MAbs that do not compete with the binding of mAb3C4 to PaCa-Ag1 are potentially useful for the “sandwich” assay, if they are of a different isotype than mAb3C4 (IgG1, κ). The new mAb should be either of the IgM or IgA isotype. This is necessary to avoid cross-reactivity of the second (indicator) Ab HRP-SαMigG with the capture mAb and mAb3C4. The second HRP-SαMigG is used to identify bound mAb3C4 in the final step of the assay that will indicate the retention in the well of PaCa-Ag1 by the capture Ab i.e. the newly defined mAb against PaCa-Ag1. Each 96-well plate may contain control wells spread throughout the plate to identify positive (purified PaCa-Ag1) as well as (Ovalbumin) negative reactions and background binding. A set of the control wells may be processed with the complete mAb3C4 and HRP-SαMIgG and TMB while other control wells will be processed with the second Ab, HRP-SαMIgG only to establish background measurement. Patient samples may be examined in triplicates using 0.05 to 0.1 ml serum, ascites, ERCP juice, or urine per well for PaCa-Ag1 protein retention.

It is possible that a mAb of a different isotype and subtype and specific for PaCa-Ag1 cannot be identified to allow the use of second HRP-labeled Ab in the assay. In this case, a commercial company may derivative the mAb3C4 directly to HRP. In this way, one will be able to use HRPA-mAb3C4 in direct measurements of the captured PaCa-Ag1 in the wells. Alternatively, FITC-mAb3C4 in a fluorophore-based assay may be used since FITC-mAb3C4 binds as well to the cell surfaces of PaCA-Ag1-positive pancreatic carcinoma cells as the unlabeled mAb3C4. In fact, FITC-mAb3C4 was used in the quantitation of PaCa-Ag1 sites establishing by FACS (see above).

In place of the above cited approaches, purified PaCa-Ag1 protein [derivatized to keyhole limpet hemocyanin (KLH) or, preferentially to an immunologically inert carrier such as high MW Ficoll MW 400,000 (Schneider et al., 1971)] may be used to generate in another animal (rabbit, goat) polyclonal PaCa-Ag1-specific Abs (pαPaCa-Ag1 Ab). The use of a pαPaCa-Ag1 Ab may be advantageous in an antigen-capture assay in that several to many anti-PaCa-Ag1 Abs may cooperate to retain the PaCa-Ag1 from the mixture of serum proteins added to the wells. It should be pointed out, however, that in the preparation of the pαPaCa-Ag1 in different animals a redistribution of low to high affinity pαPaCa-Ag1 Abs may occur according to the animals immune responses to PaCa-Ag1 protein. Purification of the pαPaCa-Ag1-IgG will not affect this situation. ELISA plates for PaCa-Ag1 prepared with the pαPaCa-Ag1-IgG obtained from different animals may give different readings on the same samples. Thus, the preparation of ELISA plates coated with pαPaCa-Ag1 IgG will require stringent quality control to correct for batch to batch differences in the pαPaCa-Ag1-IgG. Such differences can be reduced, if a large pool of pαPaCa-Ag1-IgG is generated to prepare the ELISA plates for this study. The arrangement of positive and negative controls in the 96-well ELISA plates using the pαPaCa-Ag1 Ab will be much the same as described above. MAb3C4 followed by HRP-SαMIgG can then be used as the Ab to indicate the retention of PaCa-Ag1 from positive sera.

### REFERENCES

Ahlgren J D. Epidemiology and risk factors in pancreatic cancer. Semin Oncol 23:241-250, 1996
Aisenberg, A. C., and Davis, C. (1968). The thymus and recovery from cyclophosphamide-induced tolerance to sheep erythrocytes. J. Exp. Med. 128(1), 35-46.
Aisenberg, A. C. (1967). Studies on cyclophosphamide-induced tolerance to sheep erythrocytes. J. Exp. Med. 125, 833-845.
Alanen, K. A., Joensuu, H., Klemi, P. J., and Nevalainen, T. J., (1990). Clinical Significance of nuclear DNA content in pancreatic carcinoma. J. Path. 160, 313-320.
Albert M B, Steinberg W, Henry J P. Elevated scerum levels of tumor marker CA 19-9 in acute and biliary disease. Dig Dis Sci, 33:1223-1225, 1988.
Almoguerra C, Shibata D, Forrester K, Martin J, Amheim N, Perucho M. Most human carcinomas of the exocrine pancreas contain mutant c-K-ras genes. Cell 53:549-554, 1988.
Altmann F, Schweiszer S, Weber C, Kinetic comparison of peptide: N-glycosidases F and A reveals several differences in substrate specificity. Glycoconj J 12:84-93, 1995
Amsterdam, A., and Jamieson J. D., (1974). Studies on dispersed pancreatic exocrine cells. I. Dissociation techniques and morphologic characteristics of separated cells. J. Cell Biol. 63, 1037-1056.
Anderson, R. G. and Fletcher, J. C.; N. Engl. J. Med. 1980, 303: 1293-1297: Sounding boards. Gene therapy in human beings: When is it ethical to begin?
Appert H. (1990). Composition and Production of Pancreatic Tumor Related Antigens. Int. J. Pancreatol. 7(1-3), 13-23.
Arends J W. Distribution of monoclonal antibody-defined monosialoganglioside in normal and cancerous human tissues: an immunoperoxidase study. Hybridoma 2:219-229, 1982
Argani P, Iacobuzio-Donahue C, Ryu B. Hruhan R H et al. Mesothelin is overexpressed in the vast majority of ductal adenocarcinomas of the pancreas: identification of a new pancreatic cancer marker by serial analysis of gene expression. Clin Cancer Res 7:3862-68, 2001
Audisio R. A., Veronesi P., Maisonneuve P., Chiappa, A., Andreoni, B., Bombardieri, E., and Geraghty, J. G. (1996). Clinical relevance of serological markers in the detection and follow-up of pancreatic adenocarcinoma. Surg. Oncol. 5, 49-63.
Bao, L Y, W L Thelmo, S Somnay, C Madahar, J Michl. Characterization of an acinar cell line, BMRPA.430, derived from adult rat pancreas. FASEB Journal, 8:64A, 1994.
Barbacid M. ras Genes. Annu Rev Biochem 56:779-825, 1987.
Barlogie, B., Raber, M. N., Schumann, J., Johnson, T. S., Drewinko, B., Swartzendruher, D. E., Gohde, W., Andreeft M., and Freireich, E. J. (1983). Flow cytometry in clinical cancer research. Cancer Res. 43, 3982-3997.
Baskaran, K., Laconi, S., and Reddy, M. K. (1994). Transformation of hamster pancreatic duct cells by 4-(methylnitrosamino)-1-(3-pyridyl)-1-butanone (NNK), in vitro. Carcinogenesis 15(11), 2461-2466.
Bauw, G., Van Damme, J., Puype, M., Vandekerckhove, J., Gesser, B., Ratz, G. P., Lauridsen, J. B., and Celis, J. E. (1989). Protein-electroblotting and -microsequencing strategies in generating protein data bases from two-dimensional gels. Proc. Natl. Acad. Sci. USA 86, 7701-7705.
Beaufay H, Amar-Costesec A. Methods in Membrane Biology, vol 6. Ed. Korn E D, Plenum Press, 1976
Belinsky, S. A., K. J. Nicula, W. A. Palmisano, R. Michels, G. Saccomanno, E. Gabrielson, S. B. Baylin, and J. G. Herman, 1998. Aberrant methylation of p16^{INK4a} is an early event in lung cancer and a potential biomarker for early diagnosis. Proc. Natl. Acad. Sci. USA. 95: 11891-11896.
Birkmeyer J D, Warshaw A L, Finlayson S R G et al. Relationship between hospital volume and late survival after pancreaticoduodenectomy. Surgery 126: 178-83, 1999
Borowitz M J, J Shuster, A J Caroll, M Nash, A T Look, B Camitta, D Mahoney, S J Lauer, D J Pullen. Prognostic significance of fluorescence intensity of surface marker expression in childhood B-precursor acute lymphoblastic leukemia. Blood 89:3960-3966, 1997
Bradu, S. An in vitro model of pancreatic carcinogensis: Characterization of transformation-associated biological and molecular alterations induced by the tobacco smoke carcinogen NNK in rat pancreatic BMRPA1 cells. Thesis submitted/accepted for the Doctor of Philosophy, School of Graduate Studies, SUNY-Downstate Medical Center, 11999.
Bradu, S, J R Jagdeo, B Sang, J Saukin, J Michl. Identification of transformation-associated antigen induced by the tobacco-specific carcinogen NNK in pancreatic acinar cells. Molecular Biology of the Cell, 9:490a, 1998
Branson, J. L., Graham, F. L. and Gauldie, J.; Curr. Opin. Biotechnol. 1995, 6: 590-595: The use of adenoviral vectors for gene therapy and gene transfer in vivo.
Brooks P. C., Lin J. M., French D. L., and Quigley J. P. (1993). Subtractive immunization yields monoclonal antibodies that specifically inhibit metastasis. J. Cell Biol., 122(6):1351-9.
Caldas C, Hahn S A, Hruhan R H et al Detection of K-ras mutations in the stool of patients with pancreatic adenocarcinoma and pancreatic ductal hyperplasia. Cancer Res 54:3568-3573, 1994
Carpalan-Holmstrom M, Louhim J, Stenman U H, Alfthan H, Haglund C, CEA, CA 19-9 and CA72-4 improve the diagnostic accuracy in gastrointestinal cancers. Anticancer Res. 22:2311-2316, 2002
Chang J H, Takeuchi T. Clinical significance of Span-1 antigen in the diagnosis of pancreatic cancer. J Japn Pancreas Soc 5:80-88, 1990
Chang K, Pastan I. Molecular cloning and expression of a cDNA encoding a protein detected by the K1 antibody from an ovarian carcinoma (OVCAR-3) cell line. Int J Cancer 57:90-97, 1994.
Chang K. Pastan 1. Molecular cloning of mesothelin, a differentiation antigen present on mesothelium, mesotheliomas, and ovarian cancers. Proc Natl Acad Sci. USA. 93:136-140, 1996.
Chang K, Pai L H, Batra J K, Pastan I, Willingham M C. Characterization of the antigen (CAK1) recognized by monoclonal antibody K1 present on ovarian cancers and normal mesothelium. Cancer Res 52:181-186, 1992.
Chung, S. E., (1986) in vitro transformation of human epithelial cells. Biochemica and Biophysica Acta 823, 161-194.
Colburn, N. H., W. F. Vorder Bruegge., J. R. Bates, R. H. Gray, J. D. Rossen, W. H. Kelsey, and T. Shimada, 1978. Correlation of anchorage-independent growth with tumorigenicity of chemically transformed mouse epidermal cells. Cancer Res., 38:624-634.
Curphey. T. J., C. I. Coon, B. K. Schaeffer, and D. S. Longnecker, 1987. In vivo and in vitro genotoxicity of selected compounds toward rodent pancreas. Carcinogenesis, 8:8: 1033-7.
Deissler H, F Lottspeich, M F Rajewsky. Affinity purification and cDNA cloning of rat neural differentiation and tumor cell surface antigen gp130RB13-6 reveals relationship to human and murin PC-1. J Biol Chem. 270:9849-9855, 1995
Douglas, J. T., Rogers, B. E., Roscnfeld, M. E., Michael, S. I., Feng, M. and Curiel, D. T.; Nat. Biotechnol. 1996, 14: 1574-1578: Targeted gene delivery by tropism-modified adenoviral vectors.
Elixhauser A, Halpern M T. Economic evaluations of gastric and pancreatic cancer. Hepatogastro-enterol, 46:1206-1213, 1999.
Eskelinen M, Haglund U. Developments in serologic detection of human pancreatic adenocarcinoma. Scand J Gastroenterol, 34:833-844, 1999
Friess, H., Berbrat, P., Schilling, M., Kunz, J., Korc, M, Buchler, M. W. (1996). Pancreatic cancer: the potential clinical relevance of alterations in growth factors and their receptors. J. Mol. Med. 74:35-42.
Friess H., Gassmann M., Buchler M. W. (1997). Adjuvant therapy of pancreatic cancer using monoclonal antibodies and immune response modifiers. Int. J. Panc. 21, 43-52.
Gjertsen, M. K., Bakka, A., Breivik J., and Gaudernack G. (1996). Ex vivo ras peptide vaccination in patients with advanced pancreatic cancer: results of a phase I/II study. Int. J. Cane. 65(4), 450-3.
Govil H, Reddy V, Kluskens L, Treaba D, Massarani-Wafai R. Selvaggi S, Gattuso P. Brush cytology of the biliary tract: retrospective study of 278 cases with histopathologic correlation. Diagn Cyto-pathol, 26:273-277, 2002.
Gudjonsson B. Cancer of the pancreas, 50 years of surgery. Cancer 60:2284-303, 1987
Guz, Y., Montminy, M. R., Stein, R., Leonard, J., Gamer, L. W., Wright, C. V., and Teitelman, G. (1995). Expression of murine STF-1, a putative insulin gene transcription factor, in beta cells of pancreas, duodenal epithelium and pancreatic exocrine and endocrine progenitors during ontogeny. Development 121(1), 11-18.
Hammond, A L, R K Plemper, J Zhang, U Schneider, S J Russell and R Cattaneo, 2001. Single-chain antibody displayed on a recombinant measles virus confers entry through the tumor-associated Carcinocmbryonic-Antigen. J. Virol., 75:2087-2096.
Hecht, S. S. (1996). Recent studies on mechanisms of bioactivation and detoxification of 4-(methylnitrosamino)-1-(3-pyridyl)-1-butanone (NNK), a tobacco-specific lung carcinogen. Critical Reviews in Toxicology 26(2), 163-181.
Hecht, S. S., and Hoffmann, D. (1991), 4-(Methylnitrosamino)-1-(3-pyridyl)-1-butanone (NNK), a nicotine-derived tobacco-specific nitrosamine and carcinognen for the lung and pancreas in humans. In Origins of Human Cancer: A Comprehensive Review, Cold Spring Harbor, N.Y., Cold Spring Harbor Laboratory, 745-755.
Henry J B. Clinical Diagnosis and Management by Laboratory Methods, 21^{st} edition: Edt. J. B. Henry; Elsevier, Ansterdam, 2000.
Hitt, M. M., Addison, C. L. and Graham, F. L.; Adv. Pharmacol. 1996, 40: 137-206: Human adenovirus vectors for gene transfer into mammalian cells.
Hoffman, D., Brunnemann, D. D., Prokopcczyk, B., and Djordjevic, M. V. (1994). Tobacco-specific N-nitrosamines and Areca-derived N-nitrosamines: chemistry, biochemistry, carcinogenicity, and relevance to humans. J. of Tox. and Env. Health 41, 1-52.
Horton J. Her-2 and trastuzumab in breast cancer. Cancer Control 8:103-110, 2001
Hruban, R. H., A. D. M. van Mansfeld, G. J. A. Offerhaus, D. H. J van Wecring, D. C. Allison, S. N. Goodman, T. W. Densler, K. K. Bose, J. L., Cameron, and J. L. Bos. K-ras oncogene activation in adenocaricnoma of the human pancreas. A study of 82 carcinomas using a combination of mutant-enriched polymerase chain reaction analysis and allele-specific oligonucleotide hybridization. Am. J. Pathol., 143:545-554.
Hruban, R. H., C. J. Yco, and S. E. Kern, 1998. Pancreatic cancer. The Basis of Human Cancer, Inc, Vogelstein B., Kinzler, K. W., McGrow-Hill Inc., N.Y., 603-605.
Hruban R H, van Mansfeld A D M, Offerhaus G J A, van Weering D H J, Allison D C, Goodman S N et al. K-ras oncogene activation in adenocarcinomas of the human pancreas: a study of 82 carcinomas using a combination of mutant-enriched polymerase chain reaction analysis and allele-specific oligonuclcotide hybridization. Am J Pathol 143:4682-4689, 1993
Huynh, R., S M Bradu, O H Ahkman, S Carleton, T Zheng, L.-Y Bao, J Michl., Transformation and tumor formation in BMRPA.430 pancreatic acinar cells exposed in vitro to the tobacco smoke-specific carcinogen 4-(methyl-nitros-amino)-1-(3-pyridyl)-1-butanone. Gastroenterol. 110:A400 (1996).
Israel, B. F., Pickles, R. J., Segal, D. M., Gerard, R. D. and Kenney, S. C.; J. Virol, 2001, 75(11): 5215-5221: Enhancement of adenovirus vector entry into CD70-positive B-cell lines by using a bispecific CD70-adenovirus fiber antibody.
Ito A, Sako Y, Yamasaki H, Mamuti W, Nakaya K, Nakao M, Ishikawa Y. Development of Em18-immunoblot and Em18-ELISA for specific diagnosis of alveolar echinococcosis. Acta Trop 85:173-182, 2003
Iwase H, Hotta K. Release of O-linked glycoprotein glycans by endo-alpha-N-acctyl-D-galactosa-minidase. Methods Mol Biol 14: 151-159, 1993
Jalanko, H., P. Kuusela, et al. (1984). “Comparison of a new tumour marker, CA 19-9, with alpha-fetoprotein and carcinoembryonic antigen in patients with upper gastrointestinal diseases.” J Clin Pathol 37(2): 218-22.
Jemal A, Murray T, Samuels A, Ghafoor A, Ward E, Thun M J. Cancer statistics 2003. Ca Cancer J Clin, 53:5-26, 2003
Jones, R. T., E. A. Hudson, and J. H. Resau, 1981. A review of in vitro and in vivo culture techniques for the study of pancreatic carcinogenesis. Cancer, 47:1490-1496.
Kanovsky M, Raffo A, Drew L, Rosal R, Do T, Friedman F K, Rubinstein P, Visser J. Robinson R, Brandt-Rauf P W, Michl J, Fine R L, Pincus M R; Proc Natl Acad Sci USA. 2001 Oct. 23; 98(22):12438-43: Peptides from the amino terminal mdm-2-binding domain of p53, designed from conformational analysis, are selectively cytotoxic to transformed cells.
Katz, M. E., and Mc Cormick, F. (1997) Signal transduction from multiple Ras effectors. Curr. Op. in Genet. and Dev., 7: 75-79, 1997.
Kohler G., and Milstein C. (1975). Continuous culture of fused cells secreting antibody of pre-destined specificity. Nature 256, 495-497.
Koprowski H. Steplewski Z. Mitchell K. et al. Colorecial carcinoma antigens detected by hybridoma antibodies. Somat Cell Genet 5:957-072, 1979
Kovarova, H., Stulik, J., Hochstrasser, D. F., Bures, J., Melichar, B., and Jandik, P. (1994). Two-dimensional electrophoretic study of normal colon mucosa and colorectal cancer. Appl. Theoret. Electrophoresis 4, 103-106.
Lacmmli, H U, Cleavage of structural proteins during the assembly of the head of bacteriophage T4. Nature 227:680-685, 1970.
Lagrange, P. H., Macanese, G. B., and Miller, T E (1974). Potentiation of T-cell-mediated immunity by selective suppression of antibody formation with cyclophosphamide. J. Exp. Med. 139, 1529-1539.
Leget, G. A., and Czuczman, M. S. (1998). Use of rituximab, the new FDA-approved antibody. Curr. Opin. Oncol. 10(6), 548-51.
Lee, J Y, T H Pack. Enzymatic in vitro glycosylation using peptide-N-Glycosidase F. Enzyme Microbial Technol 30:716-720, 2002
Lillemoe, Keith D, Yeo, Charles J, Cameron, John L, Pancreatic Cancer: State-of-the-Art Care. Cancer J Clin 50: 241-68, 2000
Lum, L. G. (1999). T cell-based immunotherapy of cancer: a virtual reality? CA Cancer J. Clin. 49, 74-100.
Maestranzi s, Przemioslo R, Mitchell I, Sherwood R A. The effect of benign and malignant liver disease on the tumour markers CA 19-9 and CEA. Ann Clin Biochem, 35:99-103, 1998
Magnani J, Nilsson B, Brockhaus M, et al. Amonclonal antibody-defined antigen associated with intestinal cancer is a ganglioside containing sialylated lacto-N-fucopentaose II. J Biol Chem 257:14365-14369, 1982
Marchand, M., van Baren, N., Weyanants, P I., Brichard, V., Dreno, B., Tessier, M. H., Rankin, E., Parmiani, G., Arienti, F., Humblert, Y., Bourind, A., Vanwijck, R., Lienard, D. Beauduin, M., Dietrich, P. Y., Russo, V., Derger, J., Masucci, G., Fager, E., De Greve, J., Atzpodien, J/. Brasseur, F. Coulie, P. G., van der Bruggen, P., and Boon, T. (1999). Tumor regressions observed in patients with metastatic melanolma treated with an antigenic peptide encoded by gene MAGE-3 and presented by HLA-AI. Int. J. Cancer 80(2), 219-30.
Marinova-Mutachieva, L., Goranova, I., and Goranov, I. (1990). Positive effect of cyclophosphamide on the expression of MHC class II antigens. Meth. Find. Exp. Clin Pharmacol. 12(8), 545-549.
Metzgar, R. S., Gaillard, M. T., Levine, M. T., Levine, J. S., Tuck, F. L., Bossen, E. H., and Borowitz, M. J. (1982). Antigens of human pancreatic adenocarcinoma cells defined by murine monoclonal antibodies. Canc. Res. 42, 601-608.
Nestle, I. O., Slijagic, S., Gilliet, M., Sun, Y., Grabbe, S., Dummer, R., Burg, G., and Schadendorf, D. (1998). Vaccination of melanoma patients with peptide- or tumor lysate-pulsed dendritic cells. Nat. Med. 4 (3), 328-32.
Nix G A J J, Dubbelman C, Wilson J H P et al. Prognostic implications of tumor diameter in carcinoma of the head of the pancreas. Cancer 67:529-535, 1991
Nurse, L, R Poretzky, T Zheng, L.-Y Bao, J Michl., In vitro model of a pancreatic acinus: Morphological and functional characteristics of pancreatic acini expressed by BMRPA.430 acinar cells grown on collagen-coated surfaces. Gastroenterology, 110: A422, 1996icw Group
O'Farrell P H. High resolution two-dimensional electrophoresis of proteins. J Biol Chem. 250:4007-4021 (1975).
Old, L. J. (1981). Cancer immunology: the search for specificity-G. H. A. Clowes Memorial Lecture. Cane. Res. 41, 361-375.
Pancreatic Cancer: An Agenda for Action. Report of the Pancreatic Cancer Progress Review Group (PRG). Edt.; National Cancer Institute, USPHS, pp 3, 2001.
Pantel, K., Djuric, Z., and Nakeff, A. (1990). Stein cell recovery from cyclophosphamide-induced myelosuppression requires the presence of CD4+ cells. Br. J. Haematology 75, 168-174.
Parsa, I., C. A. Foye, C. M. Cleary, and D. Hoffmann, 1986. Differences in metabolism and biological effects of NNK in human target cells. Banbury Rep., 23: 233-244 (1986).
Pegram, M. D., A. Lipton, D. F. Hayes, B. L. Weber, J. M. Baselga, D. Tripathy, D, Baly, Baughman, S. A., T. Twaddell, J. A. Glaspy, and Slamon, D. J. (1998). Phase II study of receptor-enhanced chemosensitivity using recombinant humanized anti-p185HER2/neu monoclonal antibody plus cisplatin in patients with HER2/neu-overexpressing metastatic breast cancer refractory to chemotherapy treatment. J. Clin. Oncol. 16(8), 2659-71.
Petricoin E F, Liotta L A Mass spectrometry-based diagnostics: the upcoming revolution in disease detection. Clin Chem 49:533-534, 2003
Playfair, J. H. L. (1969). Specific tolerance to sheep erythrocytes in mouse bone marrow cells. Nature 222, 882-883.
Plested J S. Coull P A, Gidney M A. (2003). ELISA. Methods Mol Med 71:243-261.
Pytowski B., Easton T. G, Valinski J. E., Calderon, T., Sun, T., Christman, J. K., Wright. S. D., and Michl, J. (1988). A monoclonal antibody to human neutrophil-specific plasma membrane antigen. J. Exp. Med. 167, 421-439.
Ranginwala, M. L Chic, J Flom, S Smith, L Chic, M Kanovsky, D Chung, P W Brandt-Rauf, Z Yamaizumi, J Michl and M R Pincus, Activation of MAP Kinases defines a mito-genic signaling pathway unique to oncogenic ras-p2. Exp Cell Res, 269:162-169, 2001
Rollhauser, C. Steinberg, W. Tumor antigens in pancreatic cancer. In: Pancreatic Cancer: Pathogenesis, Diagnosis and Treatment. Edt.: H A Reber, Humana Press, Totowa, N. J. USA. pp 137-156, 1998
Rosenberg, S A, Yang J C, Schwartzentruber D J, Hwu, P., Marincola, F. M., Topulian, S. L., Restifo, N. P., Dudley, M. E., Schwarz, S. L., Wunderlich, J. R., Parkhurst, M. R., Kawakami, Y., Seipp, C. A., Einhorn, J. H., and White, D. E. (1998). Immunologic and therapeutic evaluation of a synthetic peptide vaccine for the treatment of patients with metastatic melanoma. Nature Med 4, 321-327.
Rozenblum E, Schutte M, Goggins M, Hahn S A, Panzer S, Zahurak M, Goodman S N, Sohn T A, Hruban R H, Yeo C J, Kern S E. Tumor-suppressive pathways in pancreatic carcinoma. Cancer Res. 57:1731-4, 1997
Russell R C G. Surgical resection for cancer of the pancreas. Baillieres Clin Gastroenterol 4:889-916, 1990
Safi F, Schlosser W, Falkenreck S, Beger H G. Prognostic value of CA 19-9 serum course in pancreatic cancer. Hepatogastroenterology. 45:253-9, 1998
Saiki, R. K., Scharf, S. J., Faloona, F., Mullis, K. B., Horn, G. T., Erlich, H. A. and Arnheim, N. (1985) Science, 230, 1350.
Sambrook, J., Fritsch, E. F. and Maniatis, T.; Molecular cloning. A laboratory manual, 2^{nd} ed. Cold Spring Harbor Laboratory Press 1989, Cold Spring Harbor, N.Y.
Satake K. Takeuchi T. Comparison of CA 19-9 with other tumor markers in the diagnosis of cancer of the pancreas. Pancreas 9:720-724, 1994
Schmiedl, A., Breitling, F. and Duebel, S.; Protein Engineering 2000), 13 (10): 725-734: Expression of a bispecific dsFv-dsFv′ antibody fragment in E. coli.
Schneider, C H, J Michl, A L de Weck. Antigenicity of hapten-polysaccharides (Zur Antigenitaaet von Hapten-Polysacchariden. Eur J Immunol, 1:98-106, 1971.
Schneider C, R A Newman, D R Sutherland, U Asser, M F Greaves. A one-step purification of membrane proteins using a high efficiency immunomatrix. J Biol Chem, 257:10766-10769, 1982
Scholler N, Fu N, Ye Z, Goodman G E, Hellstrom K E, Hellstrom I. Soluble member(s) of the mesothelin/megakaryocyte potentiating factor are detectable in sera from patients with ovarian adenocarcinoma. Proc Natl Acad Sci USA 96:11531-11536, 1999
Schwartz A, G E Marti, R Poon, J W Gratama, E Fernandez-Repollet. Standardizing flow cytometry: A classification system of fluorescence standards used for flow cytometry. Cytometry 33:106-114, 1998.
Segal, D. M. and Baust, Bt, J. E. G.; J. E. Coligan, A. M. Kruisbeck, D. H. Marguiles, E. M. Shevach and W. Strober (ed), Current protocols in immunology. John Wiley & Sons, Inc, New York, N.Y. 1995. p. 12.13.11-12.13.16: Production of bispecific antibodies.
Serrano, M., A. W. Lin, M. E. McCurrach, D. Beach, and S. W. Lowe, 1997. Oncogenic ras provokes premature cell senescence associated with accumulation of p53 and p16INK4a. Cell, 88:5:593-602.
Shawler et al. (1997) Advances in Pharmacology 40:309-337. Academic Press.
Shen, R., Su, Z., Olssn, C. A., Goldstein, N. I., and Fisher, P. B. (1994). Surface-epitope masking: a strategy for the development of monoclonal antibodies specific for molecules expressed on the cell surface. J. Natl. Cancer. Inst. 86(2), 91-98.
Shi S R, Imam S A, Young L, Cote R J, Taylor C R: Antigen retrieval immunohistochemistry under the influence of pH using monoclonal antibodies. J Histochem Cytochem 43:193-201, 1995
Shin, S. I., V. H. Freedman, R. Risser, and R. Pollack, 1975. Tumorigenicity of virus-transformed cells in Nu/Nu mice is correlated specifically with anchorage independent growth in vitro. Proc. Natl. Acad. Sci. USA 72: 11:4435-39.
Srivastava, P. K. (1996). Do human cancers express shared protective antigens? Or the necessity of remembrance of things past. Semin. Immun. 8, 295-302.
Srivastava, P. K. and L. J. Old. (1988). Individually distinct transplantation antigens of chemically induced mouse tumors. Immun. Today, 9(3), 78-83.
Staretz, M. E., and S. E. Hecht. 1995. Effects of phenethyl isothiocyanate on the tissue distribution of 4-(methylnitrosamino)-(3-pyridyl)-1-butanone and metabolites in F344 rats. Cancer Res., 55:5580-88.
Steinberg, W. Clinical Utility of the CA 19-9 tumor associated antigen. Am J Gastroenterol, 85:359-355, 1990
Streit M., R. Schmidt, R. U. Hilgenfeld, E. Thiel. and E. D. Kreuser, 1996. Adhesion receptors in malignant transformation and dissemination of gastrointestinal tumors. Molecular Med., 74:253-268.
Takasaki H. Uchida E, Tempero M et al. Correlative study on expression of CA 19-9 and DUPAN-2 in tumor tissue and in serum of pancreatic cancer patients. Cancer Res. 48:1435-1438, 1988.
Tempero, M. A., E. Uchida, et al. (1987). “Relationship of carbohydrate antigen 19-9 and Lewis antigens in pancreatic cancer.” Cancer Res 47(20): 5501-3.
Tsuchiya R, Tomioka T. Izawa K et al. Collective review of small carcinomas of the pancreas. Ann Surg, 203:77-81, 1986
Turk, J. L., Parker, D., and Poulter, L. W. (1972). Functional aspects of the selective depletion of lymphoid tissue by cyclophosphamide. Immunology. 23, 493-501.
Van Benthem, J., V. J. Feron, W. R Leeman., J. W. G. M. Wilmer, E. Vermeulen, L. den Engelse, and Scherer, 1994. Immunocytochemical identification of DNA adducts, O6-methylguanine and 7-methylguanine, in respiratory and other tissues of rat, mouse, and Syrian hamster exposed to 4-(methlynitrosamino)-1-(3-pyridyl)-1-butanone. Carcinogenesis, 15:9: 2023-2029.
Von Rosen A, Linder S, Harmenberg U, Pegert S. Serum levels of CA 19-9 and CA 50 in relation to Lewis blood cell status in patients with malignant and benign pancreatic disease. Pancreas 8:160-165, 1993
Weitmann, S. D., Lark, R. H., Coney, L. R., Fort, D. W., Frasca V., Zurawski, V. R., Jr. and Kamen, B. A.; Cancer Res. 1992, 52: 3396-3401: Distribution of the folate receptor GP38 in normal and malignant cell lines and tissues.
Wickham, T. J., Segal, D. M., Roelvink, P. W., Carrion, M. E., Lizonova, A., Lee, G. M. and Kovesdi, I.; J. Virol. 1996, 70: 6831-6838: Targeted adenovirus gene transfer to endothelial and smooth muscle cells by using bispecific antibodies.
Williams C. V., McLoon, S. C., and Stechmann, C. L., (1992). Subtractive immunization techniques for the production of monoclonal antibodies to rare antigens. Biotechniques 12(6), 842-847.
Yang, Y., Joss, K. U., Su, Q., Ertl, I. C. J. and Wilson, J. M.; Gene Ther. 1996, 3: 137-144: Immune responses to viral antigens vs. transgene product in the elimination of recombinant adenovirus infected hepatocytes in vivo.
Yang, Y., Li, Q., Ertl, H. C. J. and Wilson, J. M.; J. Virol. 1995, 69: 2004-2015: Cellular and humoral immune responses to viral antigens create barriers to lung directed gene therapy with recombinant adenoviruses.
Yang, Y., Nunes, F. A., Berenesi, K., Furth, E. F., Gonezol, E. and Wilson, J. M.; Proc. Natl. Acad. Sci. 1994, 91: 4407-4411: Cellular immunity to viral antigens limits E1-deleted adenoviruses for gene therapy.
Yazawa, S., T. Asao, et al. (1988). “The presence of CA19-9 in serum and saliva from Lewis blood-group negative cancer patients.” Jpn J Cancer Res 79(4): 538-43.
Yeo, C. J. (1995). “Management of complications following pancreaticoduodenectomy.” Sur Clin North Am 75(5): 913-24.
Yeo, C. J., J. L. Cameron, et al. (1995). “Pancreaticoduodenectomy for cancer of the head of the pancreas, 201 patients.” Ann Surg 221(6): 721-31: discussion 731-3.

What is claimed is:

1. An antibody or binding portion thereof, having binding specificity to pancreatic carcinoma specific antigen 3C4-Ag, wherein said antigen is characterized by:
a molecular weight of about 43 kDa as determined by SDS-PAGE;
a pI on isoelectrofocusing of about 4.5 to about 5.0;
being unglycosylated or minimally glycosylated; and
being primarily localized on the surface of rat and human pancreatic cancer cells but not detected in normal, non-proliferating cells.

2. The antibody of binding portion thereof, of claim 1 which also binds to a soluble pancreatic carcinoma-specific antigen having a molecular weight of about 36 to about 38 kD as determined by SDS-PAGE and isolatable from sera and other bodily fluids of pancreatic cancer patients.

3. The antibody of claim 1 which is a polyclonal antibody.

4. The antibody of claim 1 which is a monoclonal antibody.

5. A murine hybridoma cell line which produces a monoclonal antibody specifically immunoreactive with the 3C4-Ag antigen of claim 1.

6. A murine hybridoma cell line which produces the monoclonal antibody of claim 1.

7. A monoclonal antibody, mAb34C, secreted by the hybridoma cell line of claim 6.

8. The monoclonal antibody mAb3C4 of claim 4 in a humanized form.

9. An antibody according to claim 1 wherein the antibody is labeled with a fluorophore, chemilophore, chemiluminecer, photosensitizer, suspended particles, radioisotope or enzyme.

10. An antibody according to claim 7 wherein the antibody is labeled with a fluorophore, chemilophore, chemiluminecer, photosensitizer, suspended particles, radioisotope or enzyme.

11. An antibody according to claim 1 wherein the antibody is conjugated or linked to a therapeutic drug or toxin.

12. The antibody of claim 1 wherein the therapeutic drug or toxin is a peptide at least about six contiguous amino acids of the amino sequence set forth in SEQ PPLSQETFSDLWKLL (SEQ ID NO:1) or an analog or derivative thereof.

13. The antibody of claim 12 wherein the penetratin sequence from antennapedia protein having the amino acid sequence KKWKMRRNQFWVKVQRG (SEQ ID NO:4) is positioned at the carboxy terminal end of the peptide.

14. An antibody according to claim 7 wherein the antibody is conjugated or linked to a therapeutic drug or toxin.

15. A method of detecting pancreatic cancer in an animal subject, said method comprising the steps of:
(a) contacting a cell, tissue or fluid sample from the subject with at least one of an antibody or binding portion thereof which specifically binds to 3C4-Ag or an immunologically active fragment thereof: the monoclonal antibody mAb34C; or an antibody which binds the epitope bound by the monoclonal antibody mAb34C; under conditions permitting said antibody to specifically bind an antigen in the sample to form an antibody-antigen complex;
(b) detecting antibody-antigen complex in the sample; and
(c) correlating the detection of elevated levels of antibody-antigen complex in the sample with the presence of pancreatic cancer.

16. A method of treating pancreatic cancer in a patient suffering therefrom which comprises administering to the patient an effective amount of an antibody or binding portion thereof which specifically binds to 3C4-Ag or an immunologically active fragment thereof, wherein said antibody or binding portion thereof is conjugated or linked to a therapeutic drug or toxin.

17. The method of claim 16 wherein said antibody is mAb3C4.

18. The method of claim 16 wherein the therapeutic drug or toxin is a peptide of at least about six contiguous amino acids of the amino sequence set forth in SEQ PPLSQETFSDLWKLL (SEQ ID NO:1) or an analog or derivative thereof.

19. A pharmaceutical composition comprising an antibody or binding portion thereof which specifically binds to 3C4-Ag, admixed with a pharmaceutically acceptable carrier.

20. The pharmaceutical composition of claim 19 wherein the antibody or binding portion thereof which specifically binds to 3C4-Ag is conjugated or linked to a therapeutic drug or toxin.
